(19) United States
(12) Patent Application Publication
(10) Pub. No.: US 2007/0135463 A1
(43) Pub. Date: Jun. 14, 2007

(54) Bicyclic heterocycles, drugs containing said compounds, the use thereof and method for preparing same

(76) Inventors: Frank Himmelsbach, Mittelbiberach (DE); Marco Santagostino, Mittelbiberach (DE); Birgit Jung, Laupheim (DE); Rainer Soyka, Biberach (DE)

Correspondence Address: BROMBERG & SUNSTEIN LLP;NORTEL NETWORKS LIMITED, 600 TECHNOLOGY PARK DRIVE, MS E65-60-103, BILLERICA, MA, 01821, US

(21) Appl. No.: 11/567,323

(22) Filed: Dec. 6, 2006

(30) Foreign Application Priority Data
Dec. 12, 2005 (EP) 05111955
Aug. 2, 2006 (EP) 06118305

(51) Int. Cl. A61K 31/517; C07D 403/02; C07D 239/94

(52) U.S. Cl. 514/266.2; 514/266.22; 514/266.4; 544/284; 544/293

(57) ABSTRACT

The present invention relates to bicyclic heterocycles of general formula 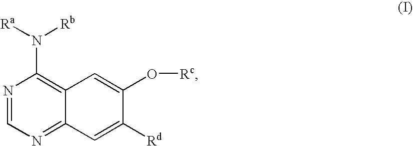 their tautomers, their stereoisomers, their mixtures and their salts, in particular their physiologically acceptable salts with inorganic or organic acids, which have valuable pharmacological properties, in particular an inhibitory action on the signal transduction mediated by tyrosine kinases, their use for the treatment of illnesses, in particular of tumoral diseases and of benign prostatic hyperplasia (BPH), of diseases of the lung and of the airways, and the preparation thereof.

The present invention relates to bicyclic heterocycles of general formula 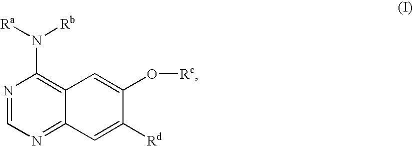
their tautomers, their stereoisomers, their mixtures and their salts, in particular their physiologically acceptable salts with inorganic or organic acids, which have valuable pharmacological properties, in particular an inhibitory action on the signal transduction mediated by tyrosine kinases, their use for the treatment of illnesses, in particular of tumoral diseases and of benign prostatic hyperplasia (BPH), of diseases of the lung and of the airways, and the preparation thereof.

In the above general formula (I)

R^{a} denotes a hydrogen atom,

R^{b} denotes a 3-chloro-4-fluoro-phenyl group or 3-ethynylphenyl group,

R^{c} denotes a group selected from among 1-methoxycarbonyl-piperidin-4-yl, 1-ethyloxycarbonyl-piperidin-4-yl, 1-trifluoroacetyl-piperidin-4-yl, cis-4-(methoxycarbonylamino)-cyclohex-1-yl, trans-4-(methoxycarbonylamino)-cyclohex-1-yl, cis-4-(ethyloxycarbonylamino)-cyclohex-1-yl, trans-4-(ethyloxycarbonylamino)-cyclohex-1-yl, cis-4-(trifluoroacetylamino)-cyclohex-1-yl, trans-4-(trifluoroacetylamino)-cyclohex-1-yl, cis-4-(N-methoxycarbonyl-N-methyl-amino)-cyclohex-1 -yl, trans-4-(N-methoxycarbonyl-N-methyl-amino)-cyclohex-1-yl, cis-4-(N-ethyloxycarbonyl-N-methyl-amino)-cyclohex-1-yl, trans-4-(N-ethyloxycarbonyl-N-methyl-amino)-cyclohex-1-yl, cis-4-(N-trifluoroacetyl-N-methyl-amino)-cyclohex-1-yl, trans-4-(N-trifluoroacetyl-N-methyl-amino)-cyclohex-1-yl, cis-4-phthalimido-cyclohex-1-yl- and trans-4-phthalimido-cyclohex-1-yl, preferably a group selected from among 1-methoxycarbonyl-piperidin-4-yl, 1-ethyloxycarbonyl-piperidin-4-yl, cis-4-(methoxycarbonylamino)-cyclohex-1-yl, trans-4-(methoxycarbonylamino)-cyclohex-1-yl, cis-4-(ethyloxycarbonylamino)-cyclohex-1-yl, trans-4-(ethyloxycarbonylamino)-cyclohex-1-yl, cis-4-(N-methoxycarbonyl-N-methyl-amino)-cyclohex-1-yl, trans-4-(N-methoxycarbonyl-N-methyl-amino)-cyclohex-1-yl, cis-4-(N-ethyloxycarbonyl-N-methyl-amino)-cyclohex-1-yl, trans-4-(N-ethyloxycarbonyl-N-methyl-amino)-cyclohex-1-yl, cis-4-phthalimido-cyclohex-1-yl and trans-4-phthalimido-cyclohex-1-yl group,

R^{d} denotes a hydrogen atom or a methoxy, ethyloxy or 2-methoxyethyloxy group, preferably a methoxy or ethyloxy group,

optionally in the form of their tautomers, racemates, enantiomers, diastereomers and mixtures thereof, as well as optionally the pharmacologically acceptable acid addition salts, solvates and hydrates thereof preferably the tautomers, racemates, enantiomers, diastereomers and mixtures thereof, as well as optionally the pharmacologically acceptable acid addition salts thereof.

The compounds of general formula (I) may be prepared for example by the following methods:
a) reacting a compound of general formula 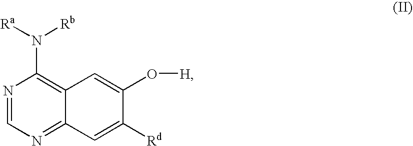
wherein
R^{a}, R^{b} and R^{d} are as hereinbefore defined, with a compound of general formula
Z¹-R^{c} (II),
wherein
R^{c} is as hereinbefore defined and Z¹ denotes a leaving group such as a halogen atom, e.g. a chlorine or bromine atom, a sulphonyloxy group such as a methanesulphonyloxy or p-toluenesulphonyloxy group or a hydroxy group.

The reaction is conveniently carried out in a solvent such as ethanol, isopropanol, acetonitrile, toluene, tetrahydrofuran, dioxane, dimethylformamide, dimethylsulphoxide or N-methylpyrrolidinone, optionally in the presence of a base such as potassium carbonate or N-ethyl-diisopropylamine, at temperatures in the range from 20° C. to 160° C., preferably at temperatures in the range from 80° C. to 140° C.

With a compound of general formula (III) wherein Z¹ denotes a hydroxy group, the reaction is carried out in the presence of a dehydrating agent, preferably in the presence of a phosphine and an azodicarboxylic acid derivative such as e.g. triphenylphosphine/diethyl azodicarboxylate, conveniently in a solvent such as methylene chloride, acetonitrile, tetrahydrofuran, dioxane, toluene or ethyleneglycoldiethylether at temperatures in the range from −50 to 150° C., but preferably at temperatures in the range from −20 to 80° C.
b) reacting a compound of general formula (IV) 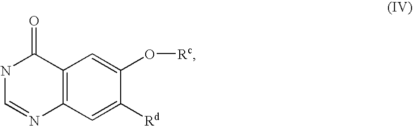
wherein R^{c} and R^{d} are as hereinbefore defined, with a halogenating agent, for example an acid halide such as thionyl chloride, thionylbromide, phosphorus trichloride, phosphorus pentachloride or phosphorus oxychloride to form an intermediate compound of general formula (V), 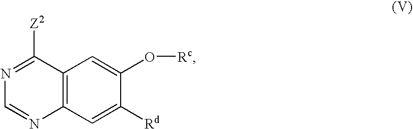
wherein R^{c} and R^{d} are as hereinbefore defined and Z² denotes a halogen atom such as a chlorine or bromine atom,
and subsequently reacting with a compound of general formula (VI)
R^{a}—NH—R^{b} (VI),
wherein R^{a} and R^{b} are as hereinbefore defined.

The reaction with the halogenating agent is optionally carried out in a solvent such as methylene chloride, chloroform, acetonitrile or toluene and optionally in the presence of a base such as N,N-diethylaniline or N-ethyl-diisopropylamine at temperatures in the range from 20° C. to 160° C., preferably from 40° C. to 120° C. Preferably, however, the reaction is carried out with thionyl chloride and catalytic amounts of dimethylformamide at the boiling temperature of the reaction mixture. It is also preferable to carry out the reaction with phosphorus oxychloride in the presence of triethylamine with acetonitrile as solvent at the boiling temperature of the reaction mixture.

The reaction of the compound of general formula (V) with a compound of general formula (VI) is conveniently carried out in a solvent such as ethanol, isopropanol, acetonitrile, dioxane or dimethylformamide, optionally in the presence of a base such as potassium carbonate or N-ethyl-diisopropylamine, at temperatures in the range from 20° C. and 160° C., preferably from 60° C. to 120° C. However, the reaction is preferably carried out in isopropanol at the boiling temperature of the reaction mixture.

Another preferred variant comprises further reacting the solution of general formula (V) obtained after the reaction with phosphorus oxychloride, in the presence of triethylamine with acetonitrile as solvent, with a solution of the compound of general formula (VI), preferably at a temperature between 20-80° C.

c) In order to prepare compounds of general formula (I) wherein R^{c} denotes a 1-methoxycarbonyl-piperidin-4-yl, 1-ethyloxycarbonyl-piperidin-4-yl, 1-trifluoroacetyl-piperidin-4-yl, cis-4-(methoxycarbonylamino)-cyclohex-1-yl, trans-4-(methoxycarbonylamino)-cyclohex-1-yl, cis-4-(ethyloxycarbonylamino)-cyclohex-1-yl, trans-4-(ethyloxycarbonylamino)-cyclohex-1-yl, cis-4-(trifluoroacetylamino)-cyclohex-1-yl, trans-4-(trifluoroacetylamino)-cyclohex-1-yl, cis-4-(N-methoxycarbonyl-N-methyl-amino)-cyclohex-1-yl, trans-4-(N-methoxycarbonyl-N-methyl-amino)-cyclohex-1-yl, cis-4-(N-ethyloxycarbonyl-N-methyl-amino)-cyclohex-1-yl, trans-4-(N-ethyloxycarbonyl-N-methyl-amino)-cyclohex-1-yl, cis-4-(N-trifluoroacetyl-N-methyl-amino)-cyclohex-1-yl, trans-4-(N-trifluoroacetyl-N-methyl-amino)-cyclohex-1-yl group, reacting a compound of general formula (VII) 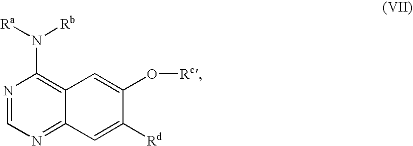
wherein R^{a}, R^{b} and R^{d} are as hereinbefore defined, and R^{c} denotes a piperidin-4-yl, cis-4-amino-cyclohex-1-yl, trans-4-amino-cyclohex-1-yl, cis-4-(methylamino)-cyclohex-1-yl or trans-4-(methylamino)-cyclohex-1-yl group,
with a corresponding acylating agent such as methyl chloroformate, ethyl chloroformate, dimethylpyrocarbonate, diethyl pyrocarbonate, trifluoroacetic anhydride or methyl trifluoroacetate.

The reaction is conveniently carried out in a solvent such as methylene chloride, acetonitrile, toluene, tetrahydrofuran, dioxane, dimethylformamide, dimethylsulphoxide or N-methylpyrrolidinone, preferably in tetrahydrofuran or dioxane, optionally in the presence of a base such as potassium carbonate, sodium hydroxide solution or N-ethyl-diisopropylamine, at temperatures in the range from −20° C. to 80° C., preferably from 0° C. to 40° C. With methyl trifluoroacetate the reaction may also be carried out in methanol.

d) In order to prepare compounds of general formula (I) wherein R^{c} denotes a cis-4-phthalimido-cyclohex-1-yl or trans-4-phthalimido-cyclohex-1-yl group, reacting a compound of general formula (VIII) 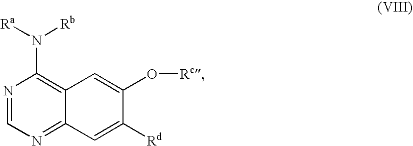
wherein R^{a}, R^{b} and R^{d} are as hereinbefore defined, and R^{c″} denotes a cis-4-amino-cyclohex-1-yl or trans-4-amino-cyclohex-1-yl group,
with phthalic anhydride or another reactive derivative of phthalic acid.

The reaction is conveniently carried out in a solvent such as acetic acid, acetonitrile, toluene, tetrahydrofuran, dioxane, dimethylformamide, dimethylsulphoxide or N-methylpyrrolidinone, optionally in the presence of a base such as potassium carbonate or N-ethyl-diisopropylamine, at a temperature in the range from 60° C. to 160° C., preferably from 80° C. to 120° C.

Preferably, however, the reaction is carried out in acetic acid at temperatures between 80° C. to 120° C.

Compounds of general formula (I) wherein R^{a}, R^{b}, R^{c} and R^{d} are as hereinbefore defined are also suitable as starting compounds for preparing corresponding quinazoline derivatives of general formula (VII) 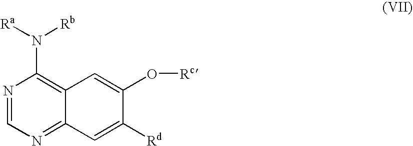
wherein R^{a}, R^{b}, R^{c} and R^{d} are as hereinbefore defined. Such compounds are described in WO 03/082290. The cleaving of the acyl groups in the group R^{c} is carried out under acid or alkaline conditions, in the case of the phthalimido group preferably with hydrazine, methylamine or ethanolamine.

The compounds of general formula (I) obtained may be resolved into their diastereomers. Thus, for example, cis/trans mixtures may be resolved into their cis and trans isomers, e.g. by chromatography.

Furthermore, the compounds of formula (I) obtained may be converted into the salts thereof, particularly for pharmaceutical use into their physiologically acceptable salts with inorganic or organic acids. Acids which may be used for this purpose include for example hydrochloric acid, hydrobromic acid, sulphuric acid, methanesulphonic acid, phosphoric acid, fumaric acid, succinic acid, lactic acid, citric acid, tartaric acid or maleic acid.

The compounds of general formulae (II) to (VIII) used as starting materials are known from the literature to some extent or may be obtained by methods known from the literature (cf. Examples I to X), optionally with the additional introduction of protecting groups.

The compounds of general formula (I) according to the invention and the physiologically acceptable salts thereof have valuable pharmacological properties, particularly an inhibiting effect on signal transduction mediated by the Epidermal Growth Factor receptor (EGF-R), whilst this may be achieved for example by inhibiting ligand bonding, receptor dimerisation or tyrosine kinase itself. It is also possible to block the transmission of signals to components located further downstream.

The biological properties of the new compounds were investigated as follows:

The inhibition of human EGF-receptor kinase was determined using the cytoplasmatic tyrosine kinase domain (methionine 664 to alanine 1186, based on the sequence published in Nature 309 (1984), 418). To do this, the protein was expressed in Sf9 insect cells as a GST fusion protein using the Baculovirus expression system.

The enzyme activity was measured in the presence or absence of the test compounds in serial dilutions. The polymer pEY (4:1) produced by SIGMA was used as the substrate. Biotinylated pEY (bio-pEY) was added as the tracer substrate. Every 100 μl of reaction solution contained 10 μl of the inhibitor in 50% DMSO, 20 μl of the substrate solution (200 mM HEPES pH 7.4, 50 mM magnesium acetate, 2.5 mg/ml poly(EY), 5 μg/ml bio-pEY) and 20 μl of enzyme preparation. The enzyme reaction was started by the addition of 50 μl of a 100 μM ATP solution in 10 mM magnesium chloride. The dilution of the enzyme preparation was adjusted so that the incorporation of phosphate into the bio-pEY was linear in terms of time and quantity of enzyme. The enzyme preparation was diluted in 20 mM HEPES pH 7.4, 1 mM EDTA, 130 mM common salt, 0.05% Triton X-100, 1 mM DTT and 10% glycerol.

The enzyme assays were carried out at ambient temperature over a period of 30 minutes and were ended by the addition of 50 μl of a stopping solution (250 mM EDTA in 20 mM HEPES pH 7.4). 100 μl were placed on a streptavidin-coated microtitre plate and incubated for 60 minutes at ambient temperature. Then the plate was washed with 200 μl of a washing solution (50 mM Tris, 0.05% Tween 20). After the addition of 100 μl of a HRPO-labelled anti-PY antibody (PY20H Anti-PTyr:HRP produced by Transduction Laboratories, 250 ng/ml) it was incubated for 60 minutes. Then the microtitre plate was washed three times with 200 μl of washing solution. The samples were then combined with 100 μl of a TMB-peroxidase solution (A:B=1:1, Kirkegaard Perry Laboratories). After 10 minutes the reaction was stopped. The extinction was measured at OD_{450 nm} with an ELISA reader. All data points were measured three times.

The data were matched using an iterative calculation using an analytical programme for sigmoid curves (Graph Pad Prism Version 3.0) with variable Hill pitch. All the iteration data released showed a correlation coefficient of more than 0.9 and the upper and lower values of the curves showed a spread of at least a factor of 5. The concentration of active substance which inhibits the activity of EGF-receptor kinase by 50% (IC₅₀) was derived from the curves. The compounds according to the invention had IC₅₀ values of less than 100 nM.

The compounds of general formula I according to the invention thus inhibit signal transduction by tyrosine kinases, as demonstrated by the example of the human EGF receptor, and are therefore useful for treating pathophysiological processes caused by hyperfunction of tyrosine kinases. These are e.g. benign or malignant tumours, particularly tumours of epithelial and neuroepithelial origin, metastasisation and the abnormal proliferation of vascular endothelial cells (neoangiogenesis).

The compounds according to the invention are also useful for preventing and treating diseases of the airways and lungs which are accompanied by increased or altered production of mucus caused by stimulation by tyrosine kinases, e.g. in inflammatory diseases of the airways such as chronic bronchitis, chronic obstructive bronchitis (COPD), asthma, bronchiectasis, allergic or non-allergic rhinitis or sinusitis, cystic fibrosis, α1-antitrypsin deficiency, or coughs, pulmonary emphysema, pulmonary fibrosis and hyperreactive airways.

The compounds are also suitable for treating inflammatory diseases of the gastrointestinal tract and bile duct and gall bladder which are associated with disrupted activity of the tyrosine kinases, such as may be found e.g. in acute or chronic inflammatory changes such as cholecystitis, Crohn's disease, ulcerative colitis, and ulcers or polyps in the gastrointestinal tract or such as may occur in diseases of the gastrointestinal tract which are associated with increased secretions, such as Ménétrier's disease, secreting adenomas and protein loss syndrome,

and also for treating inflammatory diseases of the joints, such as rheumatoid arthritis, inflammatory diseases of the skin, the eyes, in inflammatory pseudopolyps, colitis cystica profunda or pneumatosis cystoides intestinales. The compounds may also be used to treat CNS and bone marrow injuries.

Preferred fields of application which may be mentioned are inflammatory diseases of the respiratory organs or of the intestines, such as chronic bronchitis (COPD), chronic sinusitis, asthma, Crohn's disease, ulcerative colitis or polyposis of the intestine.

Particularly preferred fields of application are inflammatory diseases of the respiratory tract or lungs such as chronic bronchitis (COPD) or asthma.

In addition, the compounds of general formula (I) and the physiologically acceptable salts thereof may be used to treat other diseases caused by abnormal function of tyrosine kinases, such as e.g. epidermal hyperproliferation (psoriasis), benign prostatic hyperplasia (BPH), inflammatory processes, diseases of the immune system, hyperproliferation of haematopoietic cells, the treatment of nasal polyps, etc.

By reason of their biological properties the compounds according to the invention may be used on their own or in conjunction with other pharmacologically active compounds, for example in tumour therapy, in monotherapy or in conjunction with other anti-tumour therapeutic agents, for example in combination with topoisomerase inhibitors (e.g. etoposide), mitosis inhibitors (e.g. vinblastine), compounds which interact with nucleic acids (e.g. cis-platin, cyclophosphamide, adriamycin), hormone antagonists (e.g. tamoxifen), inhibitors of metabolic processes (e.g. 5-FU etc.), cytokines (e.g. interferons), antibodies, etc. For treating respiratory tract diseases, these compounds may be used on their own or in conjunction with other therapeutic agents for the airways, such as substances with a secretolytic (e.g. ambroxol, N-acetylcysteine), broncholytic (e.g. tiotropium or ipratropium or fenoterol, salmeterol, salbutamol) and/or anti-inflammatory activity (e.g. theophylline or glucocorticoids). For treating diseases in the region of the gastrointestinal tract, these compounds may also be administered on their own or in conjunction with substances having an effect on motility or secretion. These combinations may be administered either simultaneously or sequentially.

These compounds may be administered either on their own or in conjunction with other active substances by intravenous, subcutaneous, intramuscular, intraperitoneal or intranasal route, by inhalation or transdermally or orally, whilst aerosol formulations are particularly suitable for inhalation.

For pharmaceutical use the compounds according to the invention are generally used for warm-blooded vertebrates, particularly humans, in doses of 0.001-100 mg/kg of body weight, preferably 0.1-15 mg/kg. For administration they are formulated with one or more conventional inert carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, stearyl alcohol, carboxymethylcellulose or fatty substances such as hard fat or suitable mixtures thereof to produce conventional galenic preparations such as plain or coated tablets, capsules, powders, suspensions, solutions, sprays or suppositories.

The compounds of general formula (I) according to the invention are also suitable for preparing derivatives as described for example in WO 03/082290. For example the compound of Example 1 may be reacted with sodium hydroxide solution or potassium hydroxide solution to form 4-[(3-chloro-4-fluoro-phenyl)amino]-6-(piperidin-4-yloxy)-7-methoxy-quinazoline (cf. Method Example A).

The Examples that follow are intended to illustrate the present invention in more detail without restricting them:

Preparation of the Starting Compounds:

### EXAMPLE I

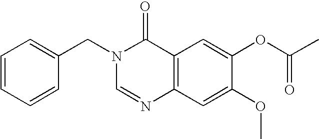

### 3-Benzyl-3,4-dihydro-4-oxo-6-acetyloxy-7-methoxy-quinazoline

169 g 3,4-dihydro-4-oxo-6-acetyloxy-7-methoxy-quinazoline, 118.8 ml benzyl bromide and 138.2 g potassium carbonate are heated in 1600 ml acetone for 8 hours to 35-40° C. The mixture is stirred for 15 hours at ambient temperature and then combined with 2000 ml of water. The suspension is cooled to 0° C., the precipitate is suction filtered, washed with 400 ml of water and 400 ml tert.-butylmethylether and dried at 50° C. The solid is dissolved in 4000 ml methylene chloride, filtered and concentrated by evaporation. The residue is suspended in tert.-butylmethylether, suction filtered and dried at 50° C.

Yield: 203 g (86% of theory)

R_{f} value: 0.80 (silica gel, methylene chloride/ethanol=9:1)

Mass spectrum (ESI⁺): m/z=325 [M+H]⁺

The following may be obtained analogously to Example I:

### (1) 3-benzyl-3,4-dihydro-4-oxo-6-acetyloxy-7-ethyloxy-quinazoline

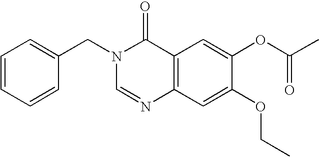

### (2) 3-benzyl-3,4-dihydro-4-oxo-6-acetyloxy-quinazoline

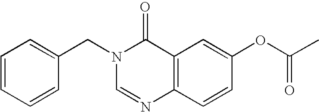

### (3) 3-benzyl-3,4-dihydro-4-oxo-6-acetyloxy-7-(2-methoxy-ethyloxy)-quinazoline

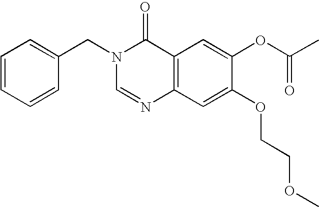

### EXAMPLE II

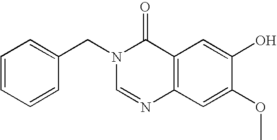

### 3-Benzyl-3,4-dihydro-4-oxo-6-hydroxy-7-methoxy-quinazoline

Method A:

168.5 g 6-hydroxy-7-methoxy-benzo[d][1,3]oxazin-4-one are dissolved in 1200 ml of toluene and 74.7 ml benzylamine are added. The mixture is refluxed for 15 hours and then cooled to ambient temperature. The precipitate is filtered off and washed with tert.-butylmethylether.

Yield 124 g (72% of theory)

Method B:

200 g 3-benzyl-3,4-dihydro-4-oxo-6-acetyloxy-7-methoxy-quinazoline are suspended in 200 ml of water and 1000 ml of ethanol. 300 ml 10N sodium hydroxide solution are added at ambient temperature and the mixture is heated to 30° C. for 1 hour. After the addition of 172 ml acetic acid and 2000 ml of water the mixture is stirred for 20 hours at ambient temperature. The precipitate is suction filtered, washed with water and acetone and dried at 60° C.

Yield: 172.2 g (98% of theory)

R_{f} value: 0.25 (silica gel, methylene chloride/ethanol=19:1)

Mass spectrum (ESI⁺): m/z=283 [M+H]⁺

The following may be obtained analogously to Example II:

### (1) 3-benzyl-3,4-dihydro-4-oxo-6-hydroxy-7-ethyloxy-quinazoline

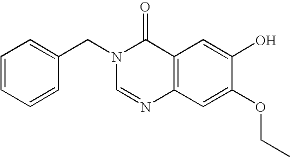

### (2) 3-benzyl-3,4-dihydro-4-oxo-6-hydroxy-quinazoline

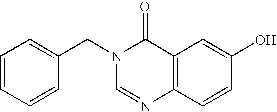

### (3) 3-benzyl-3,4-dihydro-4-oxo-6-hydroxy-7-(2-methoxy-ethyloxy)-quinazoline

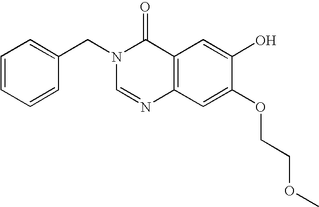

### EXAMPLE III

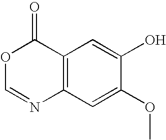

### 6-Hydroxy-7-methoxy-benzo[d][1,3]oxazin-4-one

1 g 2-amino-5-hydroxy-4-methoxy-benzoic acid (prepared by reacting methyl 2-nitro-4,5-dimethoxy-benzoate with potassium hydroxide solution to obtain 2-nitro-5-hydroxy-4-methoxy-benzoic acid potassium salt and subsequent catalytic hydrogenation in the presence of palladium on activated charcoal) and 20 ml triethyl orthoformate are heated to 100° C. for 2.5 hours. After cooling to ambient temperature the precipitate is suction filtered and washed with diethyl ether.

Yield: 0.97 g (93% of theory)

R_{f} value: 0.86 (silica gel, methylene chloride/methanol/acetic acid=90:10:1)

Mass spectrum (ESI⁺): m/z=194 [M+H]⁺

The following may be obtained analogously to Example III:

### (1) 6-hydroxy-7-ethyloxy-benzo[d][1,3]oxazin-4-one

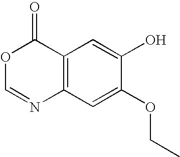

### (2) 6-hydroxy-benzo[d][1,3]oxazin-4-one

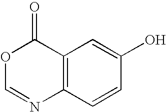

### (3) 6-hydroxy-7-(2-methoxy-ethyloxy)-benzo[d][1,3]oxazin-4-one

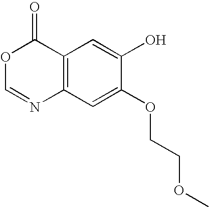

### EXAMPLE IV

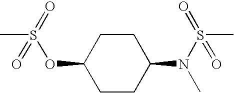

### cis-1-(Methanesulphonyloxy)-4-(N-methanesulphonyl-N-methyl-amino)-cyclohexane

Prepared by reacting cis-1-hydroxy-4-methylamino-cyclohexane with methanesulphonic acid chloride in tetrahydrofuran in the presence of triethylamine.

Mass spectrum (ESI⁺): m/z=286 [M+H]⁺

The following may be obtained analogously to Example IV:

| Example | Structure |
|---|---|
| IV(1) | 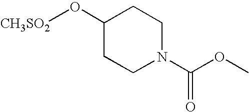 |
| IV(2) | 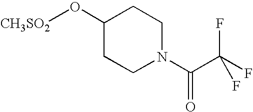 |
| IV(3) | 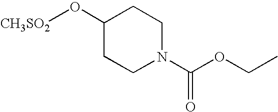 |
| IV(4) | 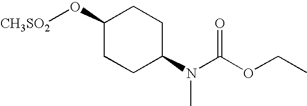 |
| IV(5) | 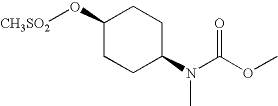 |
| IV(6) | 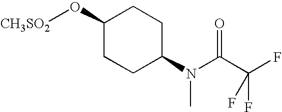 |
| IV(7) | 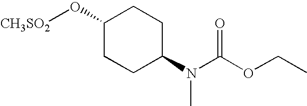 |
| IV(8) | 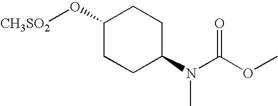 |
| IV(9) | 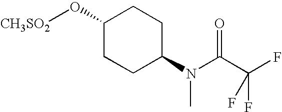 |
| IV(10) | 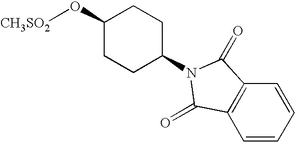 |
| IV(11) | 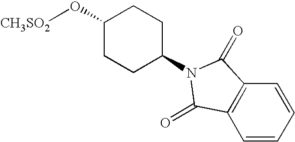 |
| IV(12) | 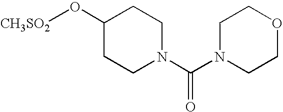 |
| IV(13) | 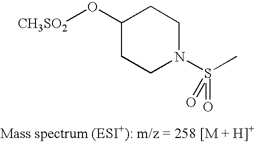 |
| IV(14) | 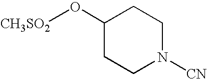 |
| IV(15) | 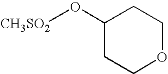 |
| IV(16) | 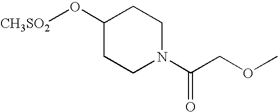 |
| IV(17) | 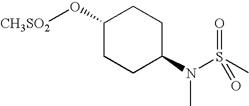 |
| IV(18) | 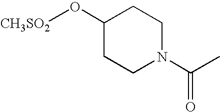 |
| IV(19) | 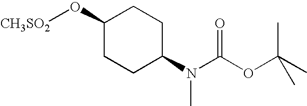 |
| IV(20) | 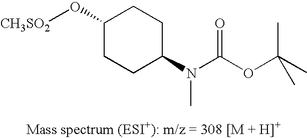 |

### EXAMPLE V

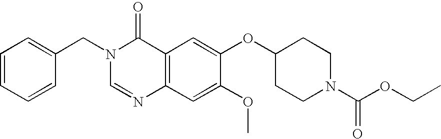

### 3-Benzyl-3,4-dihydro-4-oxo-6-(1-ethyloxycarbonyl-piperidin-4-yloxy)-7-methoxy-quinazoline

Prepared by reacting 56.46 g 3-benzyl-3,4-dihydro-4-oxo-6-hydroxy-7-methoxy-quinazoline and 62.82 g 1-ethyloxycarbonyl-4-methanesulphonyloxy-piperidine in the presence of 41.46 g potassium carbonate in 500 ml N-methyl-pyrrolidinone at 100-120° C.

Mass spectrum (ESI⁺): m/z=438 [M+H]⁺

The following may be obtained analogously to Example V:

| Example | Structure |
|---|---|
| V(1) | 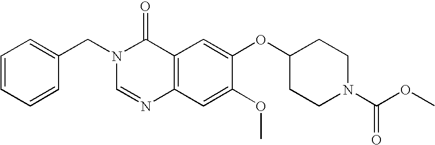 |
| V(2) | 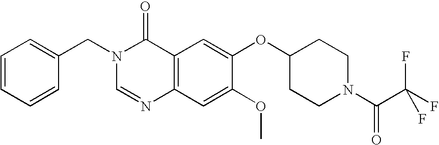 |
| V(3) | 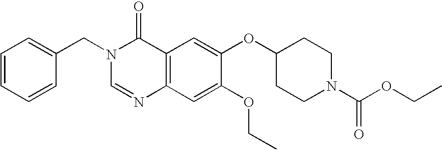 |
| V(4) | 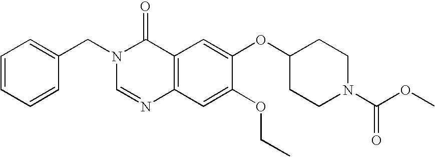 |
| V(5) | 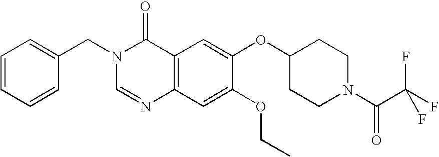 |
| V(6) | 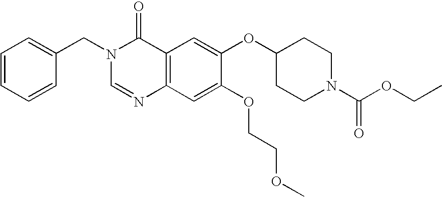 |
| V(7) | 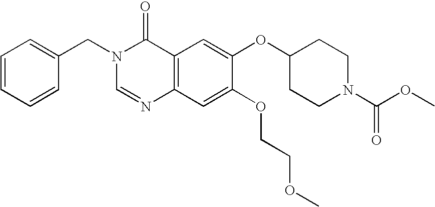 |
| V(8) | 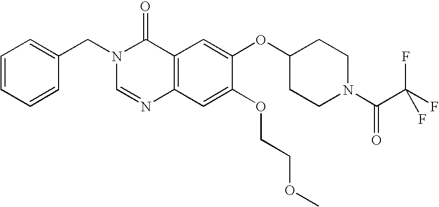 |
| V(9) | 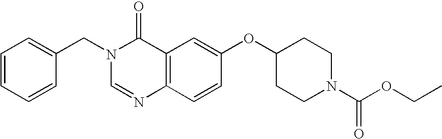 |
| V(10) | 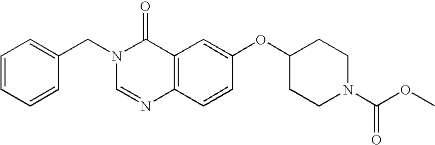 |
| V(11) | 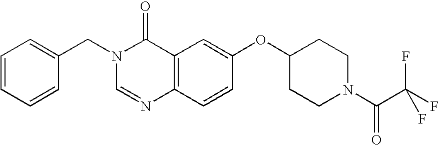 |
| V(12) | 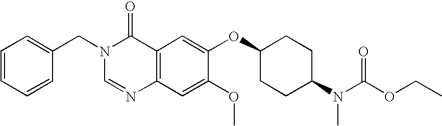 |
| V(13) | 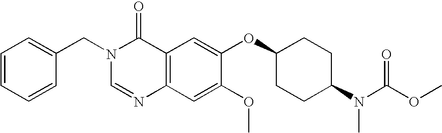 |
| V(14) | 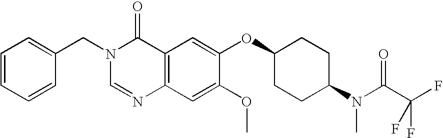 |
| V(15) | 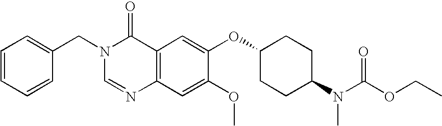 |
| V(16) | 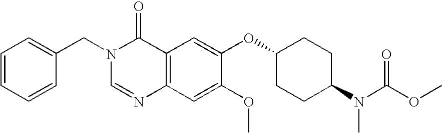 |
| V(17) | 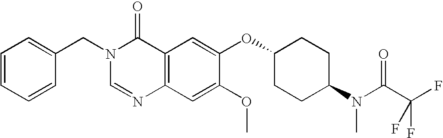 |
| V(18) | 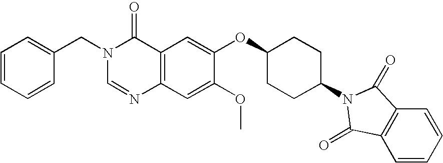 |
| V(19) | 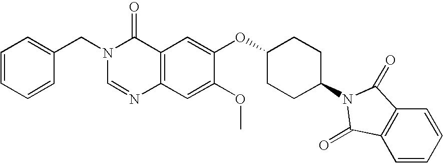 |
| V(20) | 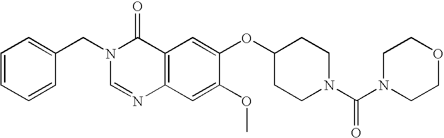 |
| V(21) | 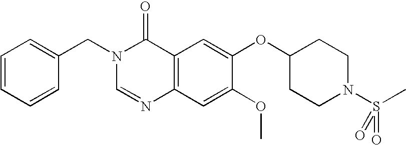 |
| V(22) | 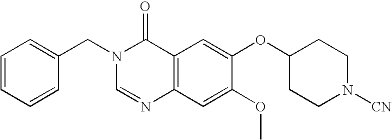 |
| V(23) | 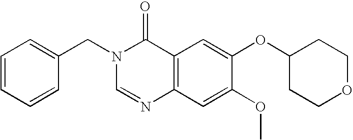 |
| V(24) | 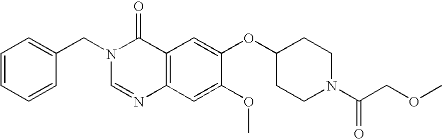 |
| V(25) | 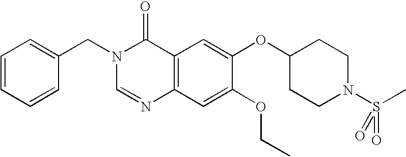 |
| V(26) | 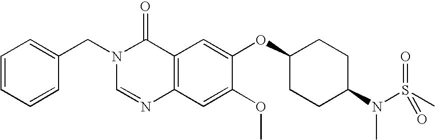 |
| V(27) | 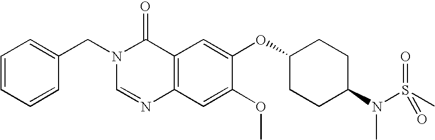 |
| V(28) | 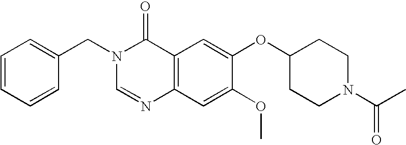 |
| V(29) | 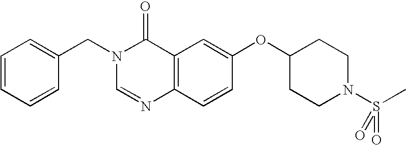 |
| V(30) | 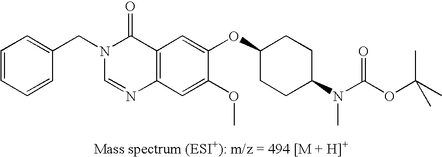 |
| V(31) | 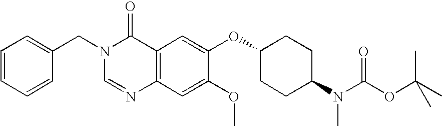 |

### EXAMPLE VI

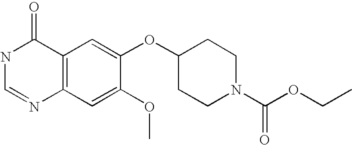

### 3,4-Dihydro-4-oxo-6-(1-ethyloxycarbonyl-piperidin-4-yloxy)-7-methoxy-quinazoline

A mixture of 58.5 g 3-benzyl-3,4-dihydro-4-oxo-6-(1-ethyloxycarbonyl-piperidin-4-yloxy)-7-methoxy-quinazoline and 600 ml glacial acetic acid are hydrogenated in the presence of 6 g palladium on activated charcoal (10% Pd) for 3 hours at 80° C. under a hydrogen pressure of 50 psi. The catalyst is suction filtered, the filtrate is concentrated down to 100 ml by evaporation and combined with 600 ml tert.-butyl-methylether. The precipitate is suction filtered and dried.

Yield: 46 g (99% of theory)

R_{f} value: 0.26 (silica gel, methylene chloride/ethanol=19:1)

Mass spectrum (ESI⁺): m/z=348 [M+H]⁺

The following may be obtained analogously to Example VI:

| Example | Structure |
|---|---|
| VI(1) | 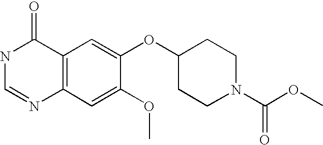 |
| VI(2) | 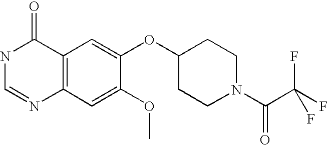 |
| VI(3) | 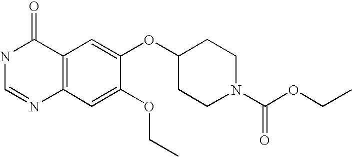 |
| VI(4) | 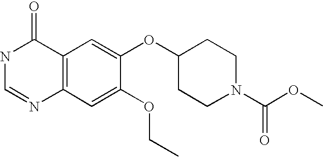 |
| VI(5) | 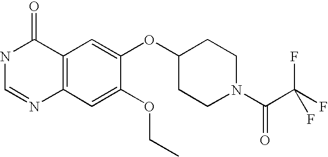 |
| VI(6) | 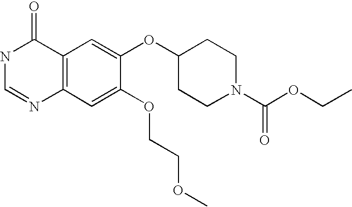 |
| VI(7) | 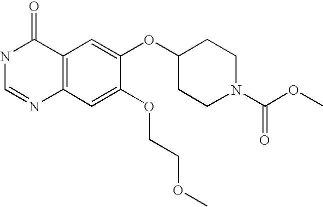 |
| VI(8) | 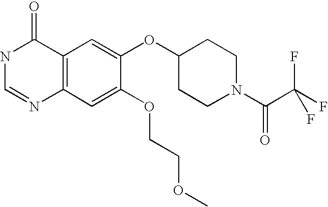 |
| VI(9) | 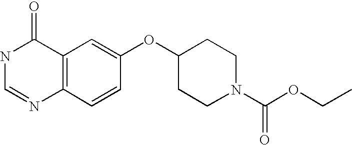 |
| VI(10) | 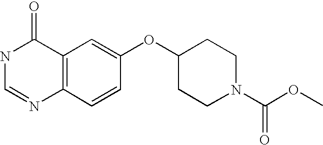 |
| VI(11) | 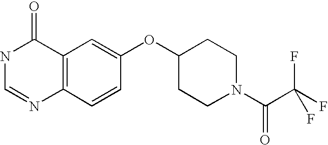 |
| VI(12) | 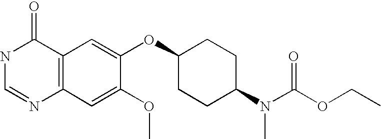 |
| VI(13) | 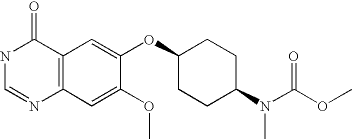 |
| VI(14) | 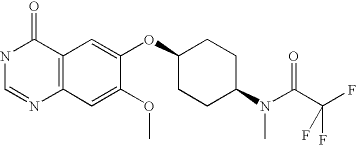 |
| VI(15) | 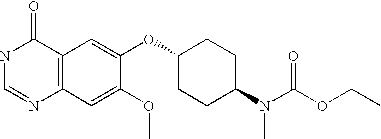 |
| VI(16) | 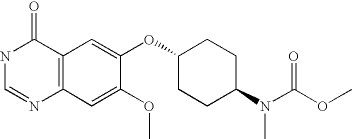 |
| VI(17) | 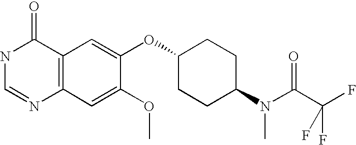 |
| VI(18) | 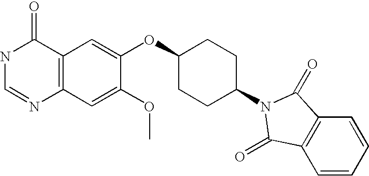 |
| VI(19) | 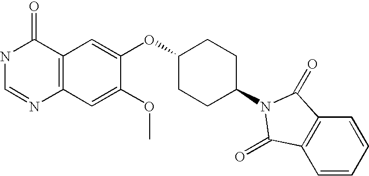 |
| VI(20) | 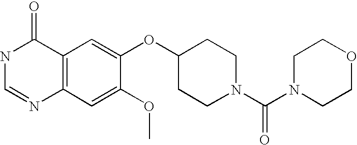 |
| VI(21) | 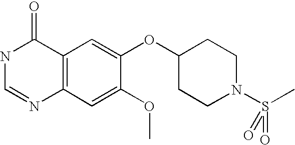 |
| VI(22) | 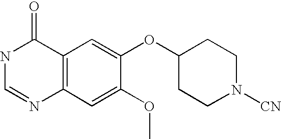 |
| VI(23) | 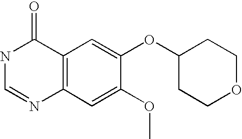 |
| VI(24) | 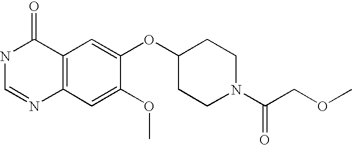 |
| VI(25) | 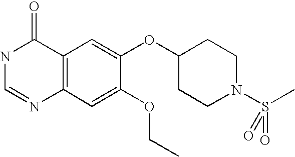 |
| VI(26) | 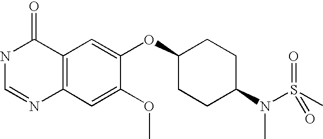 |
| VI(27) | 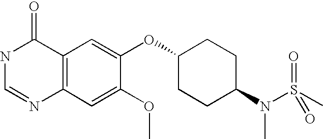 |
| VI(28) | 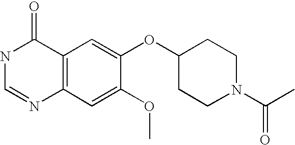 |
| VI(29) | 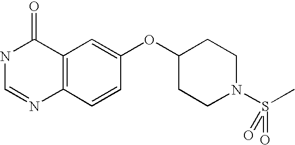 |
| VI(30) | 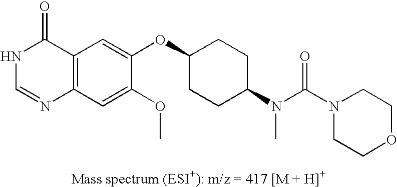 |
| VI(31) | 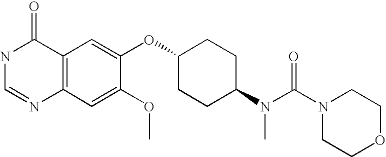 |
| VI(32) | 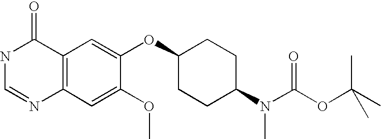 |
| VI(33) | 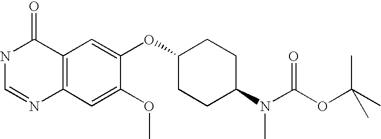 |

### EXAMPLE VII

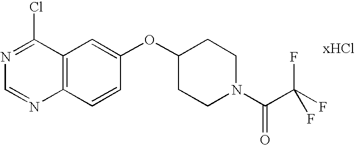

### 4-Chloro-6-(1-trifluoroacetyl-piperidin-4-yloxy)-quinazoline-hydrochloride

5.3 g 3,4-dihydro-4-oxo-6-(1-trifluoroacetyl-piperidin-4-yloxy)-quinazoline in 25 ml acetonitrile are combined with 25 ml of thionyl chloride and a few drops of dimethylformamide. The mixture is refluxed for 2 hours, concentrated by evaporation in vacuo, combined with toluene and again concentrated by evaporation. The free base may also be obtained by alkaline working up.

R_{f} value: 0.92 (silica gel, ethyl acetate)

The following may be obtained analogously to Example VII:

| Example | Structure |
|---|---|
| VII(1) | 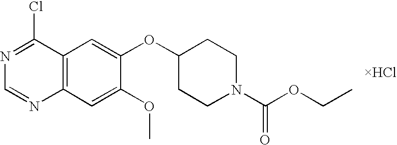 |
| VII(2) | 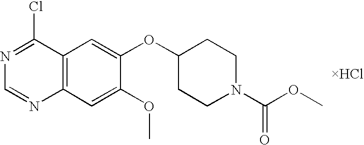 |
| VII(3) | 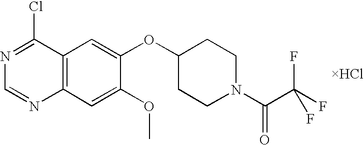 |
| VII(4) | 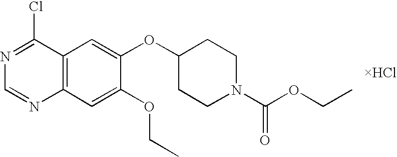 |
| VII(5) | 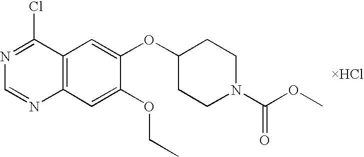 |
| VII(6) | 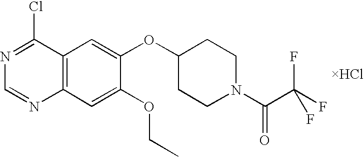 |
| VII(7) | 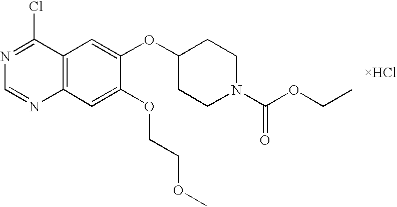 |
| VII(8) | 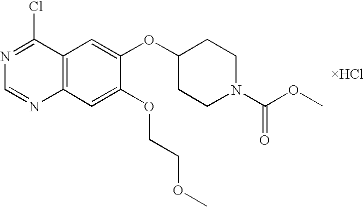 |
| VII(9) | 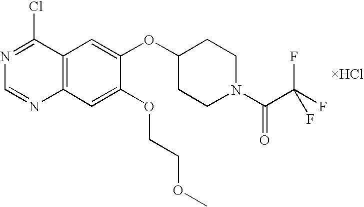 |
| VII(10) | 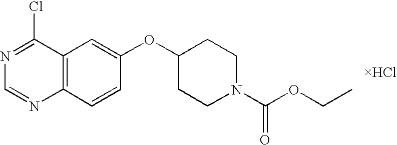 |
| VII(11) | 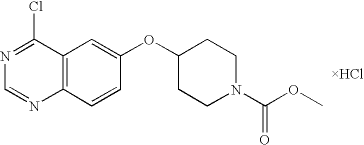 |
| VII(12) | 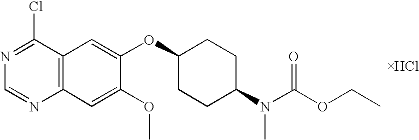 |
| VII(13) | 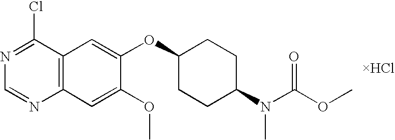 |
| VII(14) | 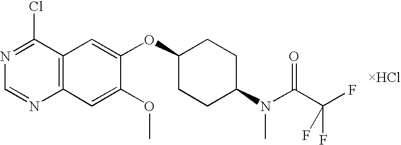 |
| VII(15) | 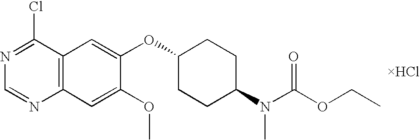 |
| VII(16) | 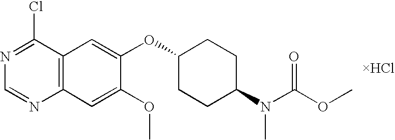 |
| VII(17) | 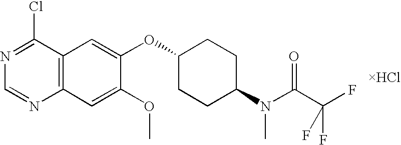 |
| VII(18) | 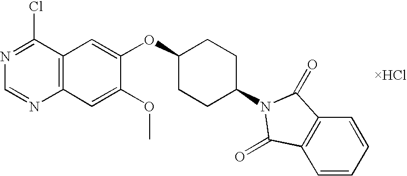 |
| VII(19) | 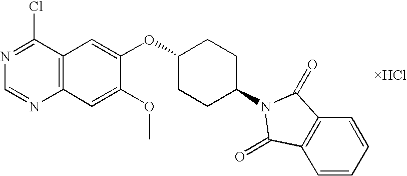 |
| VII(20) | 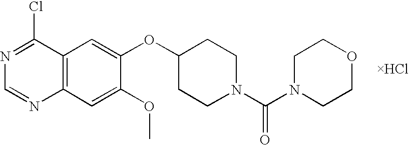 |
| VII(21) | 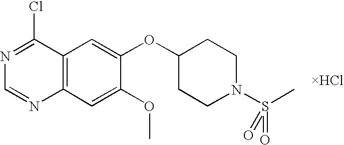 |
| VII(22) | 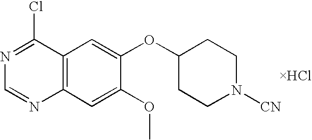 |
| VII(23) | 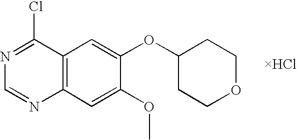 |
| VII(24) | 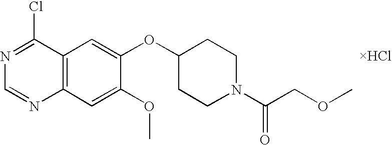 |
| VII(25) | 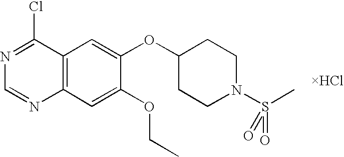 |
| VII(26) | 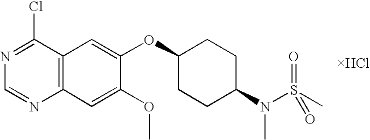 |
| VII(27) | 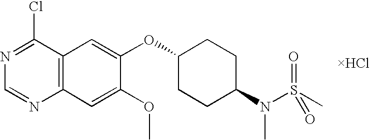 |
| VII(28) | 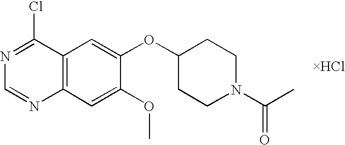 |
| VII(29) | 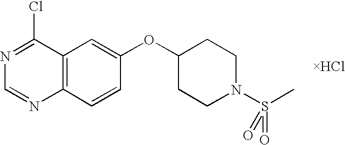 |
| VII(30) | 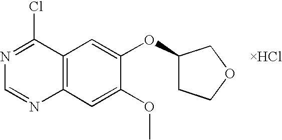 |
| VII(31) | 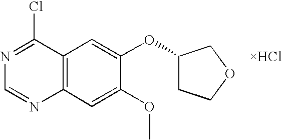 |
| VII(32) | 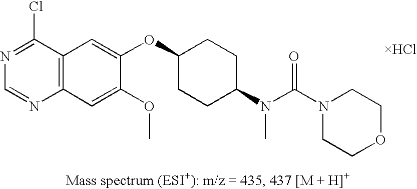 |
| VII(33) | 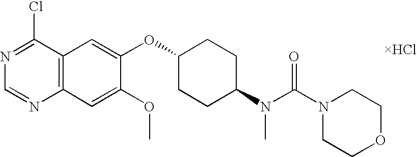 |

The free bases of the above-mentioned compounds may also be obtained by alkaline working up.

### EXAMPLE VIII

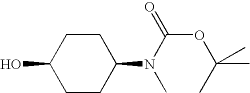

### cis-1-Hydroxy-4-(N-tert-butyloxycarbonyl-N-methyl-amino)-cyclohexane

Prepared by reacting cis-1-hydroxy-4-methylamino-cyclohexane with di-tert-butyl pyrocarbonate in ethyl acetate at ambient temperature.

Mass spectrum (ESI⁺): m/z=230 [M+H]⁺

The following may be obtained analogously to Example VIII:

| Example | Structure |
|---|---|
| VIII(1) | 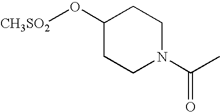 |
| VIII(2) | 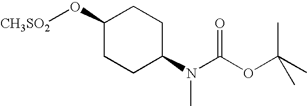 |
| VIII(3) | 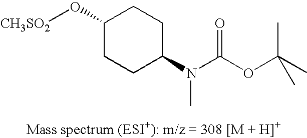 |
| VIII(4) | 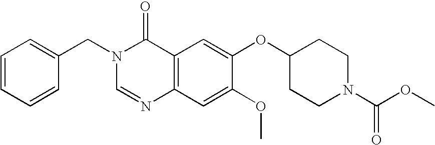 |
| VIII(5) | 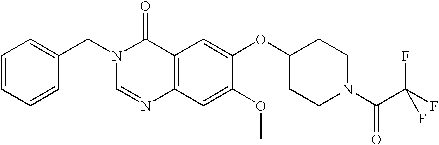 |
| VIII(6) | 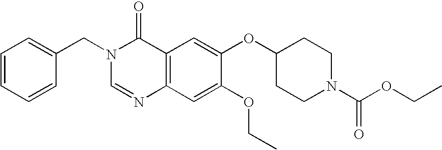 |
| VIII(7) | 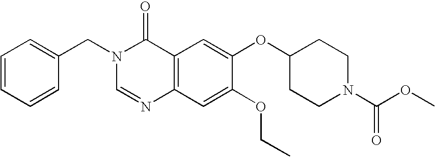 |
| VIII(8) | 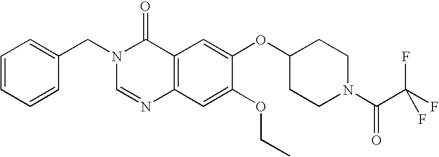 |
| VIII(9) | 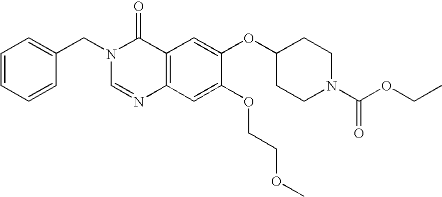 |

### EXAMPLE IX

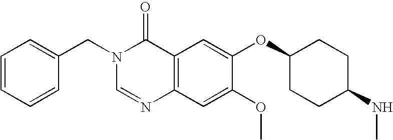

### 3-Benzyl-3,4-dihydro-4-oxo-6-(cis-4-methylamino-cyclohexan-1-yloxy)-7-methoxy-quinazoline

May be obtained by treating 3-benzyl-3,4-dihydro-4-oxo-6-{cis-4-[N-(tert-butyloxycarbonyl)-N-methyl-amino]-cyclohexan-1-yloxy}-7-methoxy-quinazoline with hydrochloric acid or trifluoroacetic acid at ambient temperature. On cleaving the tert-butyloxycarbonyl group with ethanolic or isopropanolic hydrochloric acid the hydrochloride is isolated.

Mass spectrum (ESI⁺): m/z=394 [M+H]⁺

The following may be obtained analogously to Example IX:

| Example | Structure |
|---|---|
| IX(1) | 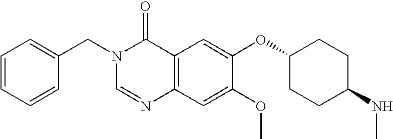 |
| IX(2) | 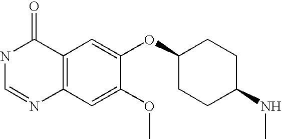 |
| IX(3) | 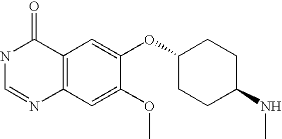 |

### EXAMPLE X

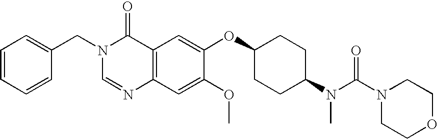

### 3-Benzyl-3,4-dihydro-4-oxo-6-(cis-4-{N-[(morpholin-4-yl)carbonyl]-N-methylamino}-cyclohexan-1-yloxy)-7-methoxy-quinazoline

May be obtained by reacting 3-benzyl-3,4-dihydro-4-oxo-6-(cis-4-methylamino-cyclohexan-1-yloxy)-7-methoxy-quinazoline in acetonitrile with (morpholin-4-yl)-carbonyl chloride in the presence of N-ethyl-diisopropylamine.

Mass spectrum (ESI⁺): m/z=507 [M+H]⁺

The following may be obtained analogously to Example X:

| Example | Structure |
|---|---|
| X(1) | 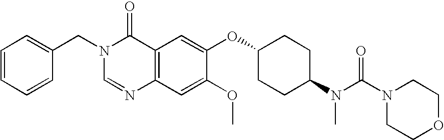 |
| X(2) | 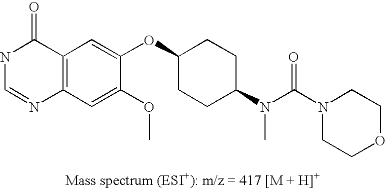 |
| X(3) | 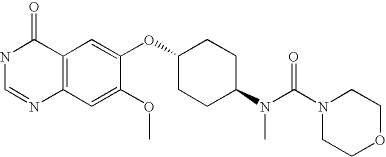 |

Preparation of the End Compounds:

### EXAMPLE 1

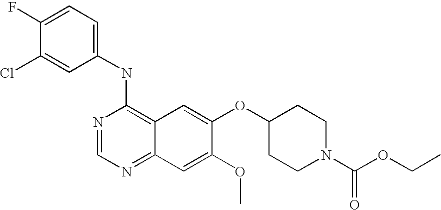

### 4-[(3-Chloro-4-fluoro-phenyl)amino]-6-(1-ethyloxycarbonyl-piperidin-4-yloxy)-7-methoxy-quinazoline

24 g of 3,4-dihydro-4-oxo-6-(1-ethyloxycarbonyl-piperidin-4-yloxy)-7-methoxy-quinazoline, 75 ml of thionyl chloride and 0.1 ml dimethylformamide are refluxed for 2 hours. The volatile parts of the reaction mixture are eliminated using the rotary evaporator and the residue is combined with 350 ml isopropanol and 23.29 g of 3-chloro-4-fluoro-aniline. The mixture is refluxed for 2.5 hours. Then 350 ml of water are added and the mixture is cooled. The solid is suction filtered and washed with water and isopropanol. The solid is suspended in 400 ml of methanol, made alkaline with concentrated aqueous ammonia. The mixture is combined with ice water and the solid is suction filtered and dried at 70° C.

Yield: 29 g (88% of theory)

R_{f} value: 0.36 (silica gel; methylene chloride/ethanol=19:1)

Mass spectrum (ESI⁺): m/z=475, 477 [M+H]⁺

The following compound is obtained analogously to Example 1:

### (1) 4-[(3-ethynyl-phenyl)amino]-6-(1-ethyloxycarbonyl-piperidin-4-yloxy)-7-methoxy-quinazoline-hydrochloride

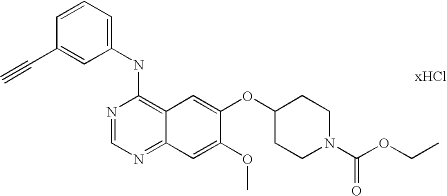

Mass spectrum (ESI⁺): m/z=447 [M+H]⁺

The following compounds may also be prepared analogously to the above-mentioned Examples and other methods known from the literature:

| Example 1 | Structure |
|---|---|
| (2) | 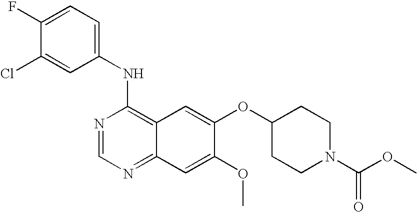 |
| (3) | 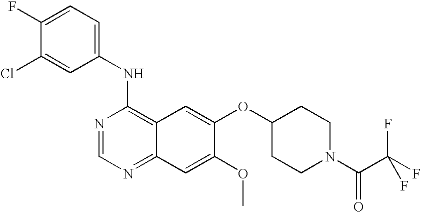 |
| (4) | 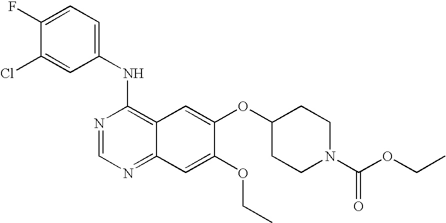 |
| (5) | 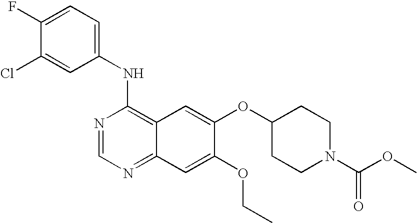 |
| (6) | 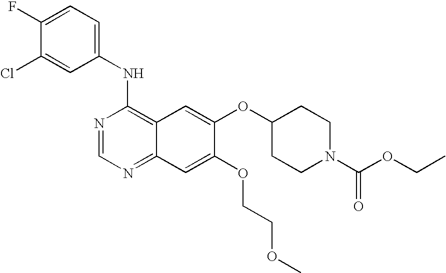 |
| (7) | 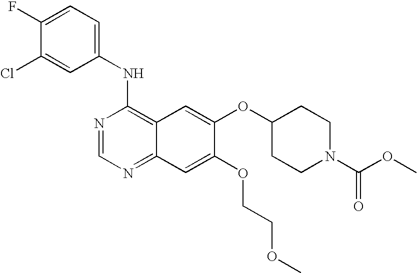 |
| (8) | 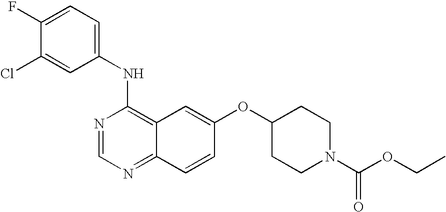 |
| (9) | 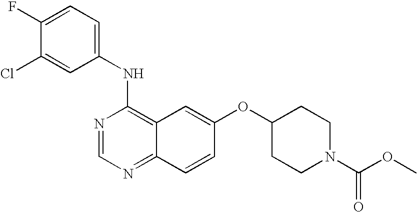 |
| (10) | 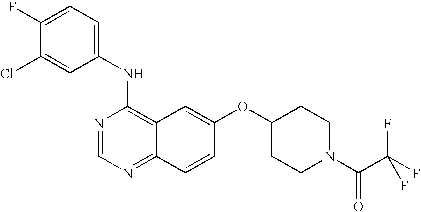 |
| (11) | 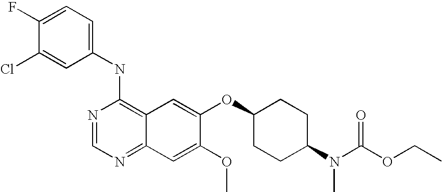 |
| (12) | 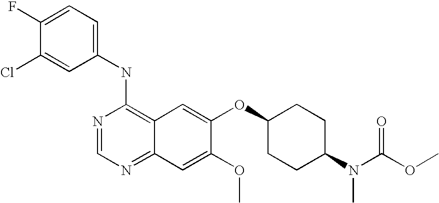 |
| (13) | 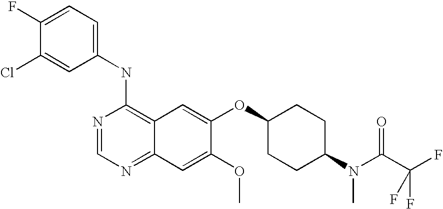 |
| (14) | 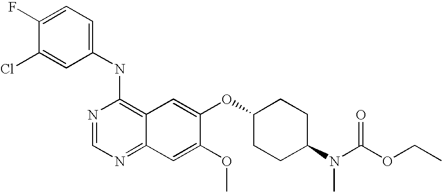 |
| (15) | 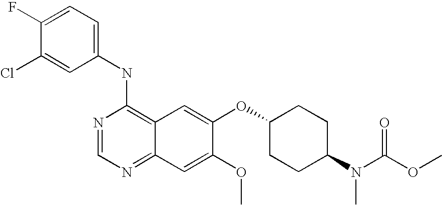 |
| (16) | 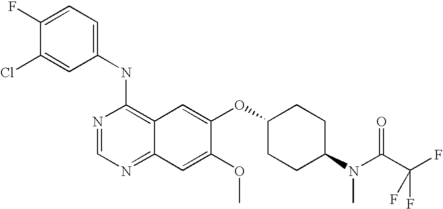 |
| (17) | 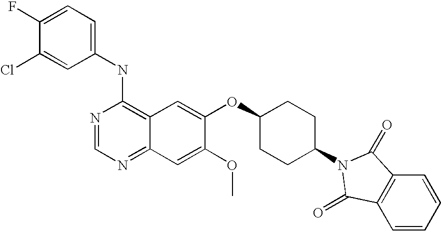 |
| (18) | 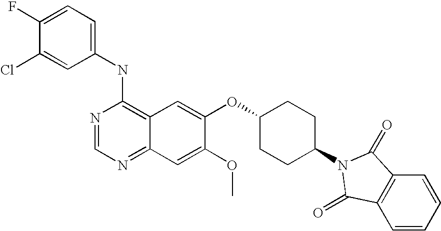 |
| (19) | 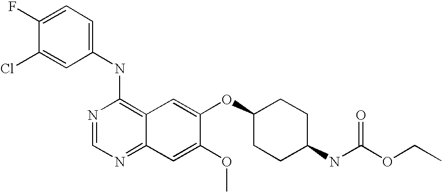 |
| (20) | 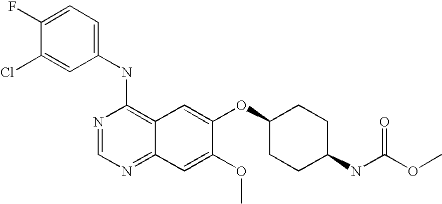 |
| (21) | 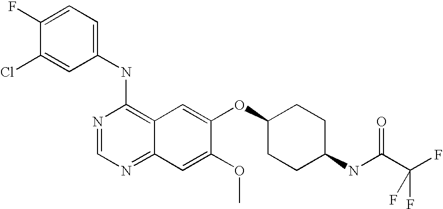 |
| (22) | 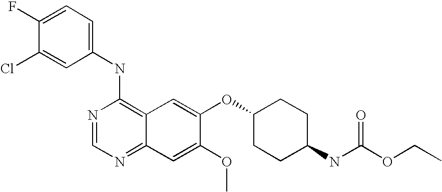 |
| (23) | 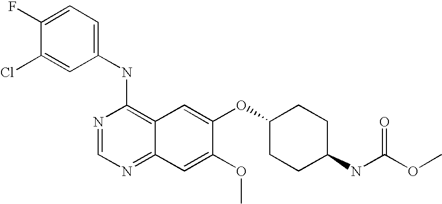 |
| (24) | 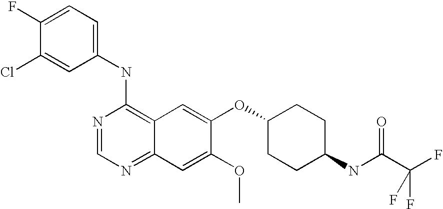 |
| (25) | 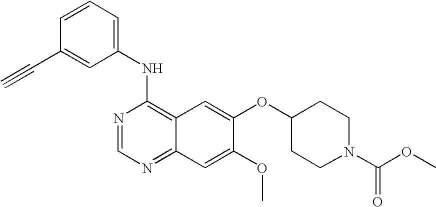 |
| (26) | 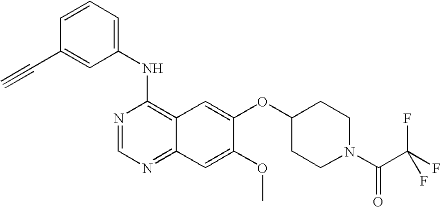 |
| (27) | 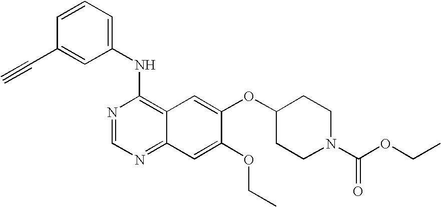 |
| (28) | 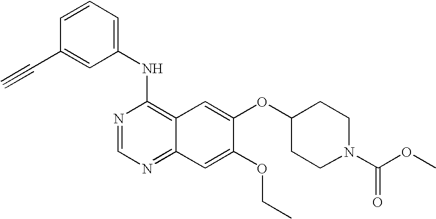 |
| (29) | 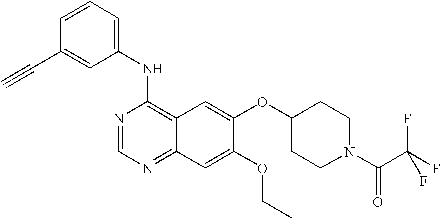 |
| (30) | 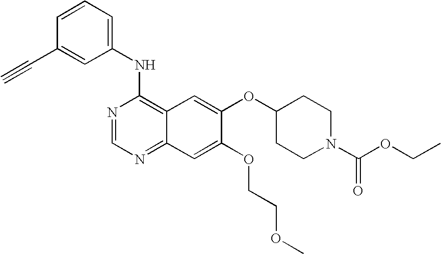 |
| (31) | 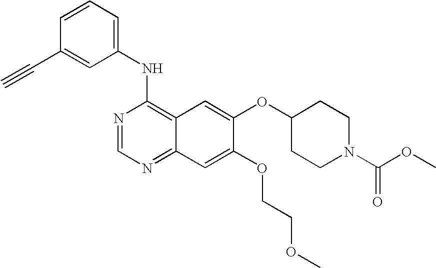 |
| (32) | 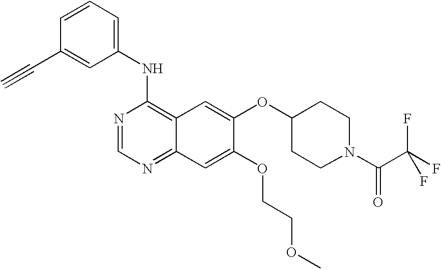 |
| (33) | 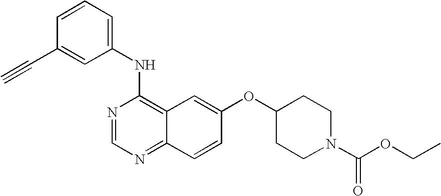 |
| (34) | 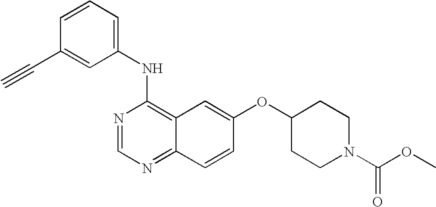 |
| (35) | 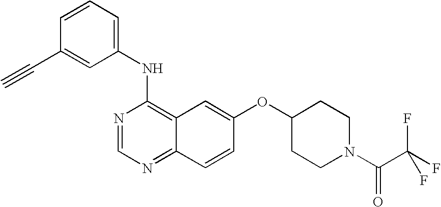 |
| (36) | 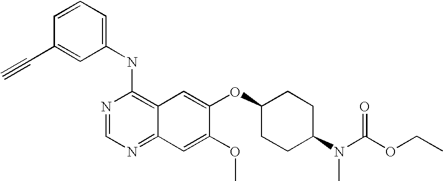 |
| (37) | 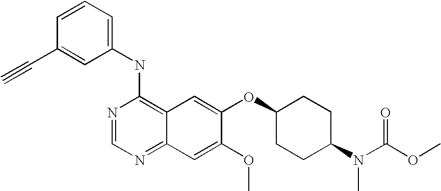 |
| (38) | 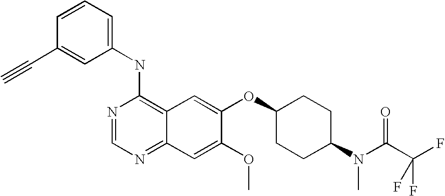 |
| (39) | 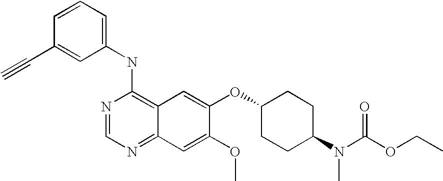 |
| (40) | 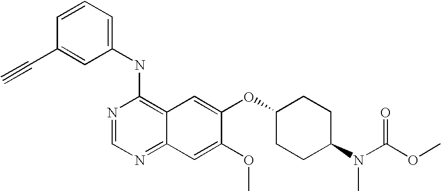 |
| (41) | 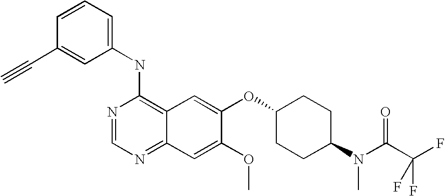 |
| (42) | 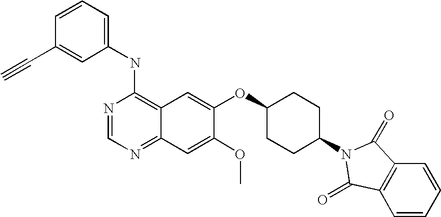 |
| (43) | 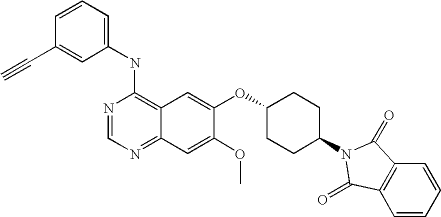 |
| (44) | 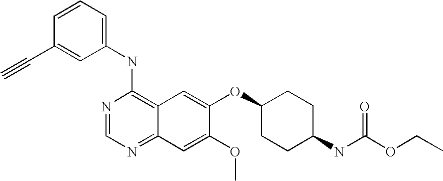 |
| (45) | 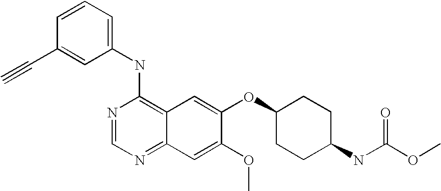 |
| (46) | 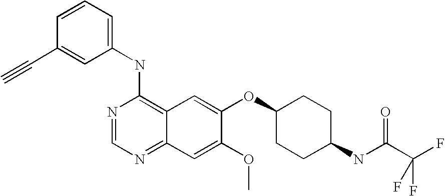 |
| (47) | 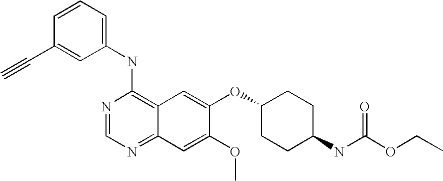 |
| (48) | 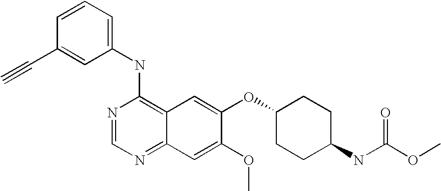 |
| (49) | 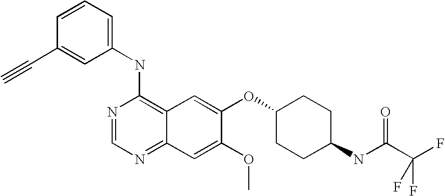 |
| (50) | 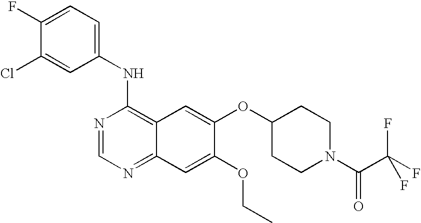 |
| (51) | 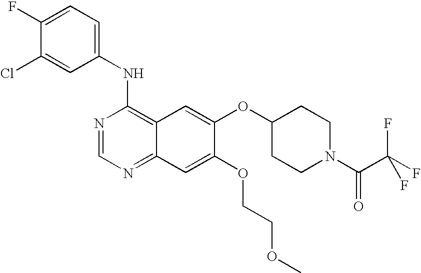 |

### EXAMPLE 2

Coated tablets containing 75 mg of active substance
1 tablet core contains:

| | | |
|---|---|---|
| active substance | 75.0 | mg |
| calcium phosphate | 93.0 | mg |
| corn starch | 35.5 | mg |
| polyvinylpyrrolidone | 10.0 | mg |
| hydroxypropylmethylcellulose | 15.0 | mg |
| magnesium stearate | 1.5 | mg |
| | 230.0 | mg |

Preparation:

The active substance is mixed with calcium phosphate, corn starch, polyvinyl-pyrrolidone, hydroxypropylmethylcellulose and half the specified amount of magnesium stearate. Blanks 13 mm in diameter are produced in a tablet-making machine and these are then rubbed through a screen with a mesh size of 1.5 mm using a suitable machine and mixed with the rest of the magnesium stearate. This granulate is compressed in a tablet-making machine to form tablets of the desired shape.
Weight of core: 230 mg
die: 9 mm, convex

The tablet cores thus produced are coated with a film consisting essentially of hydroxypropylmethylcellulose. The finished film-coated tablets are polished with beeswax.
Weight of coated tablet: 245 mg.

### EXAMPLE 3

Tablets containing 100 mg of active substance
Composition: 1 tablet contains:

| | | |
|---|---|---|
| active substance | 100.0 | mg |
| lactose | 80.0 | mg |
| corn starch | 34.0 | mg |
| polyvinylpyrrolidone | 4.0 | mg |
| magnesium stearate | 2.0 | mg |
| | 220.0 | mg |

Method of Preparation:

The active substance, lactose and starch are mixed together and uniformly moistened with an aqueous solution of the polyvinylpyrrolidone. After the moist composition has been screened (2.0 mm mesh size) and dried in a rack-type drier at 50° C. it is screened again (1.5 mm mesh size) and the lubricant is added. The finished mixture is compressed to form tablets.
Weight of tablet: 220 mg
Diameter: 10 mm, biplanar, facetted on both sides and notched on one side.

### EXAMPLE 4

Tablets containing 150 mg of active substance
Composition: 1 tablet contains:

| | | |
|---|---|---|
| active substance | 150.0 | mg |
| powdered lactose | 89.0 | mg |
| corn starch | 40.0 | mg |
| colloidal silica | 10.0 | mg |
| polyvinylpyrrolidone | 10.0 | mg |
| magnesium stearate | 1.0 | mg |
| | 300.0 | mg |

Preparation:

The active substance mixed with lactose, corn starch and silica is moistened with a 20% aqueous polyvinylpyrrolidone solution and passed through a screen with a mesh size of 1.5 mm. The granules, dried at 45° C., are passed through the same screen again and mixed with the specified amount of magnesium stearate. Tablets are pressed from the mixture.
Weight of tablet: 300 mg
die: 10 mm, flat

### EXAMPLE 5

Hard gelatine capsules containing 150 mg of active substance
Composition: 1 capsule contains:

| | | |
|---|---|---|
| active substance | 50.0 | mg |
| corn starch (dried) | approx. 80.0 | mg |
| lactose (powdered) | approx. 87.0 | mg |
| magnesium stearate | 3.0 | mg |
| | approx. 420.0 | mg |

Preparation:

The active substance is mixed with the excipients, passed through a screen with a mesh size of 0.75 mm and homogeneously mixed using a suitable apparatus. The finished mixture is packed into size 1 hard gelatine capsules.
Capsule filling: approx. 320 mg
Capsule shell: size 1 hard gelatine capsule.

### EXAMPLE 6

Suppositories containing 150 mg of active substance
Composition: 1 suppository contains:

| | | |
|---|---|---|
| active substance | 150.0 | mg |
| polyethyleneglycol 1500 | 550.0 | mg |
| polyethyleneglycol 6000 | 460.0 | mg |
| polyoxyethylene sorbitan monostearate | 840.0 | mg |
| | 2,000.0 | mg |

Preparation:

After the suppository mass has been melted the active substance is homogeneously distributed therein and the melt is poured into chilled moulds.

### EXAMPLE 7

Suspension containing 50 mg of active substance
Composition: 100 ml of suspension contain:

| | | |
|---|---|---|
| active substance | 1.00 | g |
| carboxymethylcellulose-Na-salt | 0.10 | g |
| methyl p-hydroxybenzoate | 0.05 | g |
| propyl p-hydroxybenzoate | 0.01 | g |
| glucose | 10.00 | g |
| glycerol | 5.00 | g |
| 70% sorbitol solution | 20.00 | g |
| flavouring | 0.30 | g |
| dist. water | ad 100 | ml |

Preparation:

The distilled water is heated to 70° C. The methyl and propyl p-hydroxybenzoates together with the glycerol and sodium salt of carboxymethylcellulose are dissolved therein with stirring. The solution is cooled to ambient temperature and the active substance is added and homogeneously dispersed therein with stirring. After the sugar, the sorbitol solution and the flavouring have been added and dissolved, the suspension is evacuated with stirring to eliminate air.
5 ml of suspension contain 50 mg of active substance.

### EXAMPLE 8

Ampoules containing 10 mg active substance
Composition:

| active substance | 10.0 | mg |
|---|---|---|
| 0.01 N hydrochloric acid | q.s. | |
| double-distilled water | ad 2.0 | ml |

Preparation:

The active substance is dissolved in the requisite amount of 0.01 N HCl, made isotonic with common salt, filtered sterile and transferred into 2 ml ampoules.

### EXAMPLE 9

Ampoules containing 50 mg of active substance
Composition:

| active substance | 50.0 | mg |
|---|---|---|
| 0.01 N hydrochloric acid | q.s. | |
| double-distilled water | ad 10.0 | ml |

Preparation:

The active substance is dissolved in the necessary amount of 0.01 N HCl, made isotonic with common salt, filtered sterile and transferred into 10 ml ampoules.

### EXAMPLE 10

Capsules for powder inhalation containing
5 mg of active substance
1 capsule contains:

| | | |
|---|---|---|
| active substance | 5.0 | mg |
| lactose for inhalation | 15.0 | mg |
| | 20.0 | mg |

Preparation:

The active substance is mixed with lactose for inhalation. The mixture is packed into capsules in a capsule-making machine (weight of the empty capsule approx. 50 mg).

weight of capsule: 70.0 mg

size of capsule 3

### EXAMPLE 11

Solution for inhalation for hand-held nebulisers
containing 2.5 mg active substance
1 spray contains:

| active substance | 2.500 | mg |
|---|---|---|
| benzalkonium chloride | 0.001 | mg |
| 1N hydrochloric acid | q.s. | |
| ethanol/water (50/50) | ad 15.000 | mg |

Preparation:

The active substance and benzalkonium chloride are dissolved in ethanol/water (50/50). The pH of the solution is adjusted with 1 N hydrochloric acid. The resulting solution is filtered and transferred into suitable containers for use in hand-held nebulisers (cartridges).

Contents of the container: 4.5 g

### METHOD EXAMPLE A

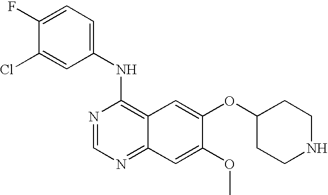

### 4-[(3-Chloro-4-fluoro-phenyl)amino]-6-(piperidin-4-yloxy)-7-methoxy-quinazoline

14.3 g of 4-[(3-chloro-4-fluoro-phenyl)amino]-6-(1-ethyloxycarbonyl-piperidin-4-yloxy)-7-methoxy-quinazoline and 15 g potassium hydroxide are heated in 250 ml isopropanol for 48 hours at reflux temperature. The reaction mixture is evaporated in vacuo down to about 50 ml and then mixed with ice water. The solid is suction filtered and recrystallised from ethyl acetate and tert.-butyl-methylether.

Yield: 9.6 g (79% of theory)

Mass spectrum (ESI⁺): m/z=403, 405 [M+H]⁺

The following may be obtained analogously to Method Example A:

| Example | |
|---|---|
| A | Structure |
| (1) | 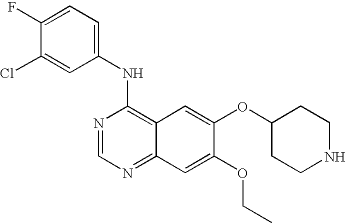 |
| (2) | 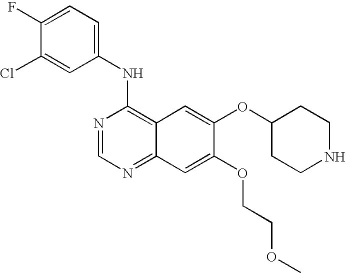 |
| (3) | 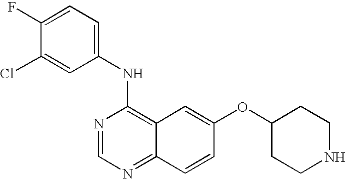 |
| (4) | 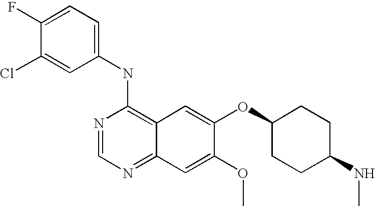 |
| (5) | 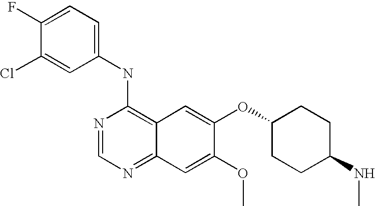 |
| (6) | 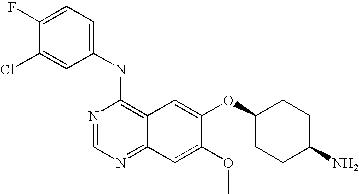 |
| (7) | 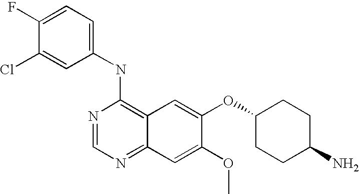 |
| (8) | 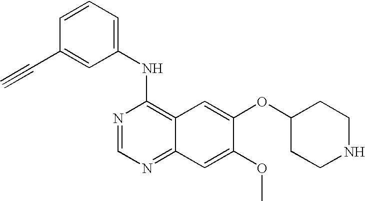 |
| (9) | 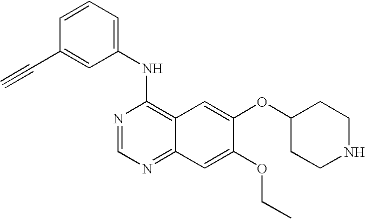 |
| (10) | 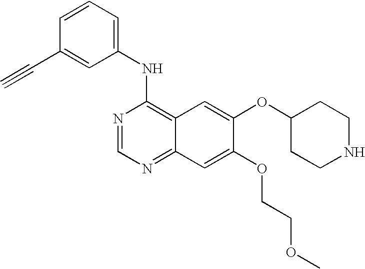 |
| (11) | 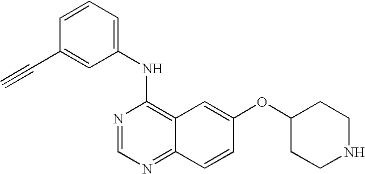 |
| (12) | 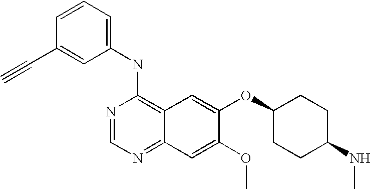 |
| (13) | 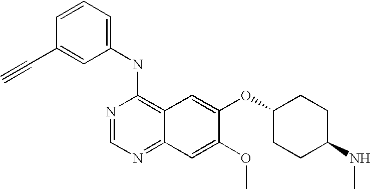 |
| (14) | 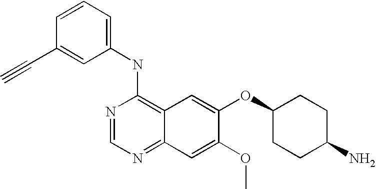 |
| (15) | 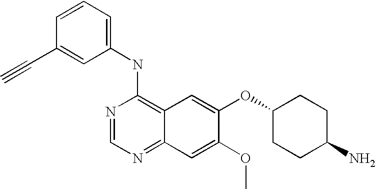 |

### METHOD EXAMPLE B

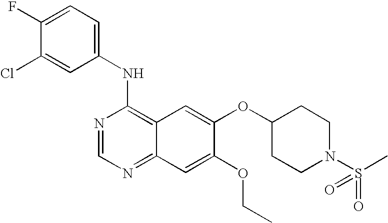

### 4-[(3-Chloro-4-fluoro-phenyl)amino]-6-(1-methanesulphonyl-piperidin-4-yloxy)-7-ethoxy-quinazoline

The title compound may be obtained from 4-chloro-6-(1-methylsulphonyl-piperidin-4-yloxy)-7-ethoxy-quinazoline-hydrochloride (Example VII(25)) by reacting with 3-chloro-4-fluoro-aniline in isopropanol at reflux temperature. The working up is carried out as described in Example 1.

The following may be obtained analogously to Method Example B:

| Example B | Structure |
|---|---|
| (1) | 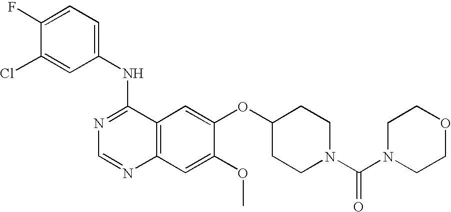 |
| (2) | 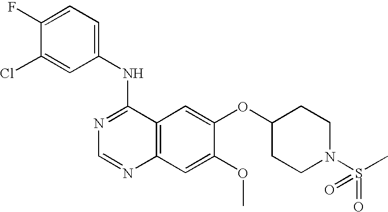 |
| (3) | 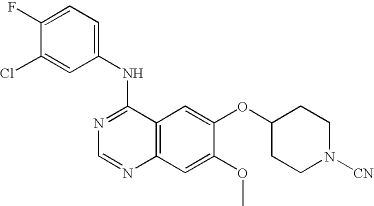 |
| (4) | 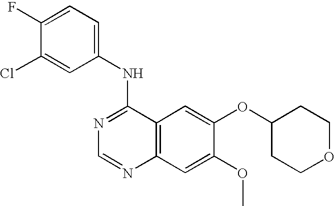 |
| (5) | 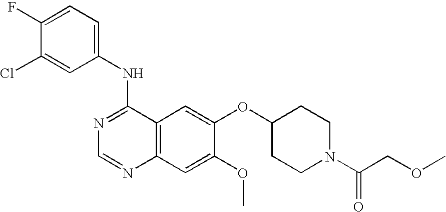 |
| (6) | 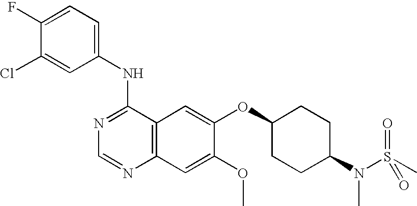 |
| (7) | 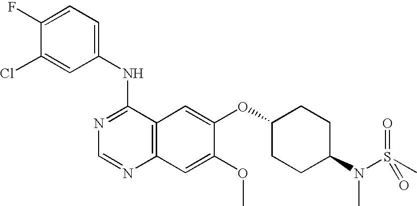 |
| (8) | 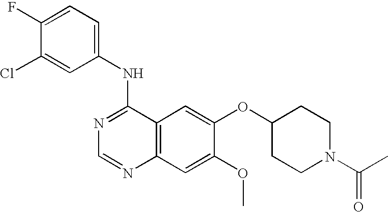 |
| (9) | 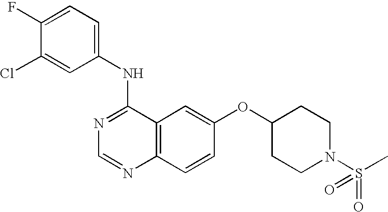 |
| (10) | 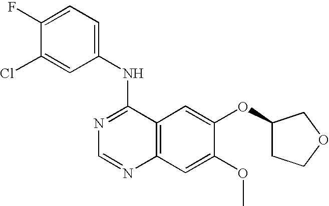 |
| (11) | 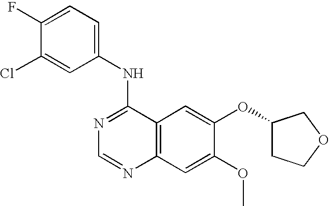 |
| (12) | 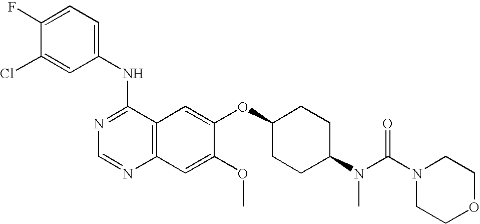 |
| (13) | 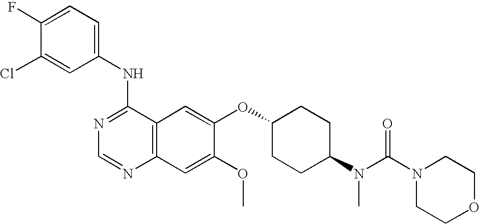 |
| (14) | 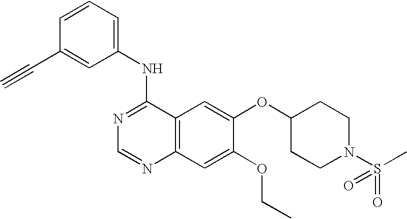 |
| (15) | 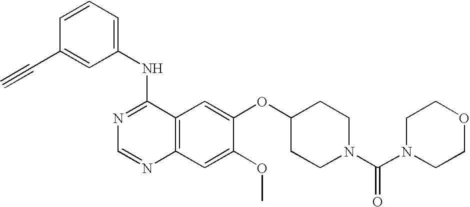 |
| (16) | 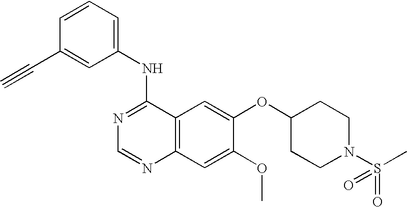 |
| (17) | 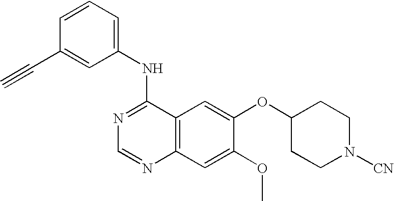 |
| (18) | 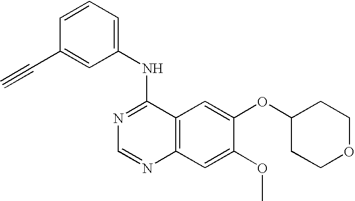 |
| (19) | 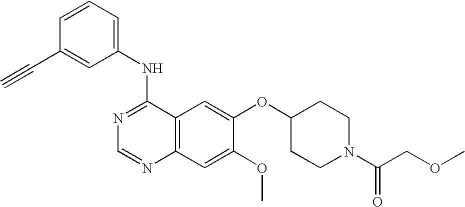 |
| (20) | 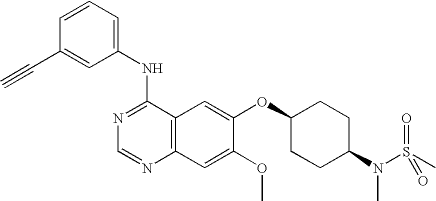 |
| (21) | 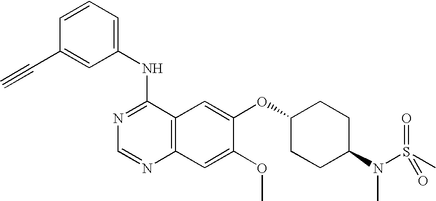 |
| (22) | 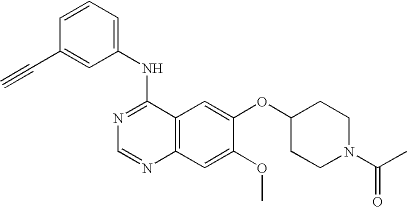 |
| (23) | 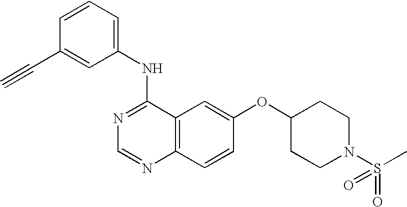 |
| (24) | 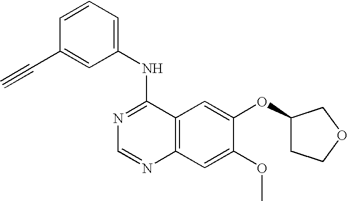 |
| (25) | 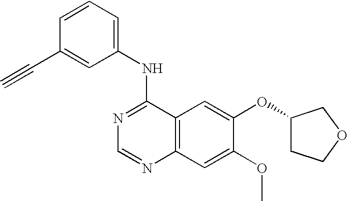 |
| (26) | 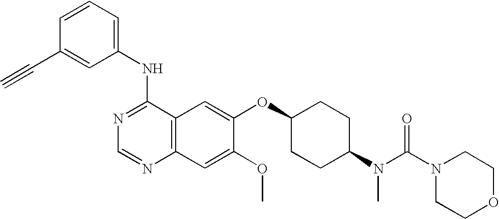 |
| (27) | 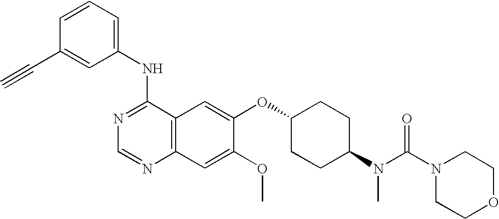 |

### METHOD EXAMPLE C

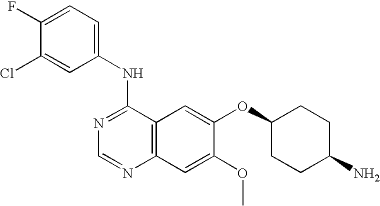

### 4-[(3-Chloro-4-fluoro-phenyl)amino]-6-(cis-4-amino-cyclohexan-1-yloxy)-7-methoxy-quinazoline

The title compound may be obtained from 4-[(3-chloro-4-fluoro-phenyl)amino]-6-(cis-4-phthalimido-cyclohexan-1-yloxy)-7-methoxy-quinazoline (Example 1(17)) by treatment with methylamine or ethanolamine.

The following may be obtained analogously to Method Example C:

| Example | |
|---|---|
| C | Structure |
| (1) | 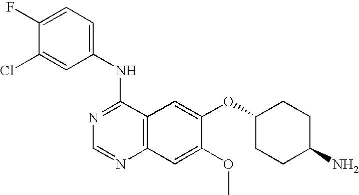 |
| (2) | 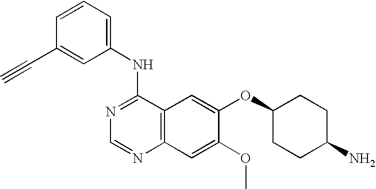 |
| (3) | 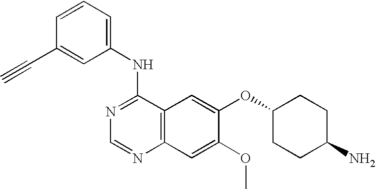 |

What is claimed is:

1. A compound of the formula (I) 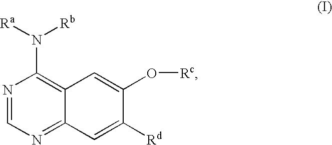
wherein
R^{a} denotes a hydrogen atom,
R^{b} denotes a 3-chloro-4-fluoro-phenyl group or 3-ethynylphenyl group, R^{c} denotes a group selected from among 1-methoxycarbonyl-piperidin-4-yl, 1-ethyloxycarbonyl-piperidin-4-yl, 1-trifluoroacetyl-piperidin-4-yl, cis-4-(methoxycarbonylamino)-cyclohex-1-yl, trans-4-(methoxycarbonylamino)-cyclohex-1-yl, cis-4-(ethyloxycarbonylamino)-cyclohex-1-yl, trans-4-(ethyloxycarbonylamino)-cyclohex-1-yl, cis-4-(trifluoroacetylamino)-cyclohex-1-yl, trans-4-(trifluoroacetylamino)-cyclohex-1-yl, cis-4-(N-methoxycarbonyl-N-methyl-amino)-cyclohex-1-yl, trans-4-(N-methoxycarbonyl-N-methyl-amino)-cyclohex-1-yl, cis-4-(N-ethyloxycarbonyl-N-methyl-amino)-cyclohex-1-yl, trans-4-(N-ethyloxycarbonyl-N-methyl-amino)-cyclohex-1-yl, cis-4-(N-trifluoroacetyl-N-methyl-amino)-cyclohex-1-yl, trans-4-(N-trifluoroacetyl-N-methyl-amino)-cyclohex-1-yl, cis-4-phthalimido-cyclohex-1-yl and trans-4-phthalimido-cyclohex-1-yl group,
R^{d} denotes a hydrogen atom, a methoxy, ethyloxy or 2-methoxyethyloxy group,
or a tautomer or salt thereof.

2. A compound of the formula (I) according to claim 1,
wherein
R^{a}, R^{b} and R^{d} may have the meanings specified, and
R^{c} denotes a group selected from among 1-methoxycarbonyl-piperidin-4-yl, 1-ethyloxycarbonyl-piperidin-4-yl, cis-4-(methoxycarbonylamino)-cyclohex-1-yl, trans-4-(methoxycarbonylamino)-cyclohex-1-yl, cis-4-(ethyloxycarbonylamino)-cyclohex-1-yl, trans-4-(ethyloxycarbonylamino)-cyclohex-1-yl, cis-4-(N-methoxycarbonyl-N-methyl-amino)-cyclohex-1-yl, trans-4-(N-methoxycarbonyl-N-methyl-amino)-cyclohex-1-yl, cis-4-(N-ethyloxycarbonyl-N-methyl-amino)-cyclohex-1-yl, trans-4-(N-ethyloxycarbonyl-N-methyl-amino)-cyclohex-1-yl, cis-4-phthalimido-cyclohex-1-yl and trans-4-phthalimido-cyclohex-1-yl group,
or a tautomer or salt thereof.

3. A physiologically acceptable salt of a compound according to claim 1 or 2.

4. A pharmaceutical composition comprising a compound according to claim 1 to 2 or a physiologically acceptable salt thereof and one or more inert carriers and/or diluents.

5. A method for the treatment of benign or malignant tumours, for the treatment of diseases of the airways and lungs and for the treatment of diseases of the gastrointestinal tract and the bile duct and gall bladder which comprises administering a therapeutically effective amount of a compound according to claim 1 to 2 or a physiologically acceptable salt thereof.
